(19) United States
(12) Patent Application Publication
(10) Pub. No.: US 2013/0142878 A1
(43) Pub. Date: Jun. 6, 2013

(54) PEPTIDE PARTICLE FORMULATION

(71) Applicant: FLOW PHARMA, INC, East Palo Alto, CA (US)

(72) Inventors: Reid M. RUBSAMEN, Alamo, CA (US); David Earl HECKERMAN, Santa Monica, CA (US)

(73) Assignee: FLOW PHARMA, INC., East Palo Alto, CA (US)

(21) Appl. No.: 13/691,234

(22) Filed: Nov. 30, 2012

Related U.S. Application Data
(60) Provisional application No. 61/565,686, filed on Dec. 1, 2011.

(51) Int. Cl. A61K 9/14; A61K 49/00

(52) U.S. Cl. CPC A61K 9/14
USPC 424/490; 424/400; 424/184.1

(57) ABSTRACT

A composition as disclosed is comprised of a plurality of groups of particles. The particles are comprised of a biocompatible polymer which may be a co-polymer such as PLGA combined with a peptide of a sequence of interest, e.g. a sequence which corresponds to a sequence presented on a surface of a cell infected with a virus. A plurality of different groups of particles are provided in the formulation wherein the particles within any single group include peptides of identical amino acid sequence. The particles are sized such that they are sufficiently large so as to prevent more than the contents of a single particle from being presented to a single immune system cell.

### CROSS-REFERENCE

This application claims the benefit of U.S. Provisional Application No. 61/565,686, filed Dec. 1, 2011, which application is incorporated herein by reference.

### FIELD OF THE INVENTION

This invention relates generally to the field of peptide particles which form formulations comprised of groups of particles. The particles of a single group of particles consist only of the same compound, such as a single peptide species.

### BACKGROUND OF THE INVENTION

The term vaccine derives from Edward Jenner's 1796 use of the term cow pox (Latin variola vaccine, adapted from the Latin vaccīn-us, from vacca cow), which, when administered to humans, provided them protection against smallpox.

The 20th century saw the introduction of several successful vaccines, including those against diphtheria, measles, mumps, and rubella. Major achievements included the development of the polio vaccine in the 1950s and the eradication of smallpox during the 1960s and 1970s. Maurice Hilleman was the most prolific of the developers of the vaccines in the twentieth century. As vaccines became more common, many people began taking them for granted. However, vaccines remain elusive for many important diseases, including malaria and HIV. Vaccines may be dead or inactivated organisms or purified products derived from them.

There are several types of vaccines currently in use. These represent different strategies used to try to reduce risk of illness, while retaining the ability to induce a beneficial immune response. Considerable efforts have been made to develop an HIV vaccine.

A cell infected with HIV virus has many distinct epitopes on its surface. Each epitope can be targeted by the cellular immune response mediated by T-lymphocytes. These T-lymphocytes become sensitized to specific epitopes by exposure to antigens brought to the T-cells by antigen presenting cells (e.g macrophages).

HIV vaccines have been developed to direct cellular immunity mechanisms toward a blood borne HIV virus by sensitizing T-cells, via antigen presenting cells (APCs) exposed to the vaccine, to suites of epitopes on the surface of cells infected with the virus.

Vectors used to introduce vaccines into the cellular immunity pathways have included adenovirus vectors. A problem with traditional vaccine approaches to treating patients already infected with HIV has been the fact that adenovirus vectors tend to activate CD4+ T-cells which in turn can potentially make pre-existing HIV infection more virulent. Another problem with HIV vaccine designs, in general, has been that the end result is to target large suites of epitopes on the surface of the cells infected with the virus, possibly targeting epitopes which could actually worsen various pathological aspects of the HIV infection.

Until recently, little was known about the specific effect of targeting specific epitopes on the surface of cells infected with the HIV virus. Each time a vaccine vector is given to a person with HIV disease, a number of epitopes are targeted, and a number of immune response parameters are measured. Associating a specific response with a specific epitope has been essentially impossible from an analysis of a single vaccine administration. Data from the administration of multiple vaccines to multiple sets of HIV infected subjects with corresponding cellular immune responses could, in theory, allow the effects of the individual epitopes to be de-convolved, essentially through a very computational intensive cross-correlation exercise.

Recent work in the field has brought modern super-computer technology to bear on this problem, resulting in a list of putative individual pathogen-relevant effects of individual epitopes on the surface of cells infected with the HIV virus.

Results from immunization with antigen-containing PLGA microspheres made from a double-emulsion process utilizing organic solvents have been mixed, however, perhaps owing to the fact that the solvent systems and shear forces used in such microsphere fabrication processes can cause protein conformational changes that may interfere with the antigen-presenting event.

Peptides injected into the lymphatic system can be taken up by APCs, thereby producing an immune response. If a single APC takes up more than one antigen and simultaneously presents multiple antigens to T-cells, this may result in a cellular immune response wherein the response is targeted to only one of the antigens that was presented.

Methods for relieving the effects of immunodominance are described in published US patent application 20080260780, entitled “Materials And Methods Relating To Improved Vaccination Strategies”; US patent application 20090269362, entitled “Method for Controlling Immunodominance”; and US patent application 20100119535, entitled “Compositions and Methods for Immunodominant Antigens.”

### SUMMARY OF THE INVENTION

A composition is disclosed which is comprised of a plurality of groups of particles, which may be substantially spherical particles, comprised of a polymer and an antigen, e.g. a peptide, lipid, glycolipid, phospholipid, polysaccharide, etc. All of the particles in all of the groups have a narrow particle size range of about 10 μm±20% to about 20 μm±20% in diameter. The size range can be such that only a single particle can be consumed by an antigen presenting cell, by which is usually intended a professional antigen presenting cell. For example, the particle size may be in a range of 12 microns to 18 microns in diameter. The particles sizes may be 15 microns in diameter±20%. The size of the particles in a given formulation may be substantially the same size, i.e. ±20% or ±10%, ±2%, or ±1% in diameter.

Each particle comprises one or more antigenic compounds, including without limitation peptides, where the antigenic compounds in a particle are of identical species or sequence. When peptides are used they are generally small and should be at least 8 and not more than 20 amino acids, not more than 15, or not more than 11 or 12 amino acids in length. Thus, the peptide size range may be 8-12 amino acids particularly epitopes for presentation by class I MHC proteins. However, where epitopes for presentation by class II MHC proteins are present, the peptides may have a broader size range, e.g. 8 or more amino acids in length, or 8-30 amino acids.

In one embodiment of the invention, peptides present in or on any one particle are identical to all other peptides on that particle, i.e. the peptides have the same amino acid sequence and there may be no other peptides in that particle with a different amino acid sequence; other compounds such as lipids, polysaccharides, etc. consist of a single species. In another embodiment, a particle comprises (i) one species of peptide that provides an epitope for class I presentation, and (ii) one species of peptide that provides an epitope for class II presentation. In certain of such embodiments, a given particle is comprised of an inert polymer such as PEG, and comprises two different species of peptides where one species provides a class I epitope that generates a specific immune response and the other peptide species provides a class II epitope that enhances or amplifies the immune response generated by the class I epitope.

The particles may be provided as a group, i.e. a composition or formulation of a plurality of particles with a single antigen species. Alternatively a composition or formulation is provided comprising a cocktail of particles with a plurality of antigen species, with the proviso that each particle contains only a single species of antigen.

In one embodiment the composition of the invention does not include a conventional adjuvant because particles of the composition may stimulate the immune system and as such act as an adjuvant.

In another embodiment the composition comprises an adjuvant in a form chosen from (a) inert biocompatible particles with no other components inside; (b) inert biocompatible particles with only ink inside; and (c) inert biocompatible particle with a peptide presented by a class II peptide inside.

In another embodiment of the invention a conventional vaccine formulation is combined with any or all of the adjuvant (a), (b) and/or (c) above and administered without a conventional adjuvant.

Formulations of the invention can be administered as vaccines to prevent an infection or as a treatment for a disease.

Formulations of the invention may be administered by any type of injection on nasally.

Formulations of the invention can act as B-cell vaccines as well as T-cell vaccines.

Compositions or groups of interest for manufacturing purposes, in kits, and the like may comprise a single antigen species, such as a single peptide species. Compositions and formulations for therapeutic purposes, e.g. for use in a method of treatment, generally include at least two antigen species. Formulations for generating an immune response may include 2, 3, 4, 5, 10, 20, 30, 40, 50 or more antigen species and an equivalent number of groups of particles.

The composition containing the plurality of groups of particles may be a vaccine formulation which may have the particles in a dry form to which a liquid carrier can be added to form a suspension or emulsion (e.g. injectable or viscous nasal spray), or the liquid carrier may be present and the liquid carrier may be a pharmaceutically acceptable injectable carrier.

A composition of the invention can include any combination of the compositions outlined in the claims below. A particular composition of the invention which includes the components of the first three claims below would be a composition which reads as follows:

A composition, comprising:

a first group of particles comprising a biocompatible polymer and a first active ingredient consisting only of a first peptide;

a second group of particles comprising a biocompatible polymer and a second active ingredient consisting only of a second peptide different from the first peptide wherein the second peptide has an amino acid sequence consisting of 8 to 12 amino acids; and

a third group of particles comprising a biocompatible polymer and a third active ingredient consisting only of a third peptide different from both the first and second peptides; and

wherein the biocompatible polymer is selected from the group consisting of poly(lactic-co-glycolic acid) (PLGA), polycaprolactone, polyglycolide, polylactic acid, poly-3-hydroxybutyrate;

wherein each particle in the first group, the second group and the third group is substantially spherical, and has a diameter in a range of from 10 microns±20% to 25 microns±20%.

In addition, the compositions of the invention can include a composition as described above with one or more adjuvants. The adjuvants may be included in a liquid carrier which the particles are present or may be coated onto the particles or incorporated into the particles. It may be useful to include toll-like receptor agonists which include CpG and MPL which is monophosphoryl lipid A. The term CPG is further described below.

CpG oligodeoxynucleotides (or CpG ODN) are short single-stranded synthetic DNA molecules that contain a cytosine “C” followed by a guanine “G”. The “p” refers to the phosphodiester backbone of DNA, however some ODN have a modified phosphorothioate (PS) backbone. When these CpG motifs are unmethlyated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is constitutively expressed only in B cells and plasmacytoid dendritic cells (pDCs) in humans and other higher primates.

The toll-like receptors can stimulate antigen presentation by dendrocytes. Toll receptors such as MPL are less active versions of LPS which is a lipid polysaccharide. The LPS causes signs and symptoms of sepsis. The LPS promotes phagocytosis. An aspect of the invention includes incorporating CpG inside the sphere and placing the MPL in solution which the spheres are suspended in as described in the examples. The CpG is included inside the cell in order to promote antigen presentation once the sphere has been phagosytosed.

An aspect of the invention is the formulation for delivering the peptide containing microspheres as described here for vaccination where a toll-like receptor agonist to promote antigen presentation is incorporated into the sphere and a toll-like receptor agonist to promote phagocytosis is added to the solution in which the spheres are suspended.

In another aspect of the invention such a formulation is produced wherein the toll-like receptor agonist to promote antigen presentation is CpG in the sphere and a toll-like receptor agonist to promote phagocytosis is MPL.

Another aspect of the invention is a nasal formulation containing compositions as encompassed by the claims. Such nasal formulations for the delivery of vaccines can be produced using excipient materials known to those skilled in the art such as disclosed within U.S. Pat. No. 6,391,318 (incorporated herein by reference) as well as the patents and publications cited in the '318 patent all of which are incorporated herein by reference.

The invention also includes a method of treating a subject such as vaccinating a subject whereby a formulation of the invention is injected into an individual in order to elicit an immune response.

The method of the invention comprises administering to a human patient a composition comprising (a) a first group of particles comprising a biocompatible polymer and a first chemical species, and (b) a second group of particles comprising a biocompatible polymer and a second chemical species, different from the first chemical species. The method is characterized by the use of particles in both the first and second group of particles which are substantially spherical in shape and have a diameter of from 10 microns±20% to 25 microns±20% or 12 microns to 22 microns in diameter±20% or about 17 microns in diameter±20%. The method is further characterized by being the initial exposure of the patient to the chemical species or antigens. More specifically the patient has not been previously exposed to such antigens. Thus, the method of the invention is not intended to be a vaccination booster within a more general method where the patient has previously been primed or exposed to a plurality of epitopes. Thus, the compositions of the invention are generally not intended for use as a “booster” to supplement the immune response of an earlier exposure to an antigen or plurality of antigens.

An aspect of the invention is that antigen presenting cells take up a single particle of the invention, which particle comprises a single antigenic species; or a single species that provides an epitope for class I presentation. Because only a single antigenic species is present, problems with immunodomination are reduced or eliminated. Accordingly, the immune system will produce a plurality of immune responses (e.g., multiple cytotoxic T-cell clones; antibodies; etc.) in response to the different antigenic species presented in different particles. This end result can be achieved by using a formulation as described here, which is comprised of groups of particles wherein each group of particles consists of only one type of antigenic particle. The result can also be obtained by a methodology whereby a formulation consisting of a first antigenic compound which is injected at a first point in time followed by an injection of a formulation consisting of a second antigenic compound at a second point in time after the first point in time. Additional steps in the process may be carried out with a third, fourth, fifth, etc. antigenic compound administered at a third, fourth, fifth, sixth, etc. point in time. The end result is to obtain multiple different immune responses with multiple different cytotoxic T-cells or multiple different antibodies which bind to multiple different antigens and thereby reduce or eliminate the immunodominance issue with respect to a human immune system. This methodology requires multiple administrations which are not required when the formulation is comprised of several different groups of particles wherein each particle consists of only a single antigenic compound. Either the formulation of multiple groups or the step by step methodology using one group with each administration can be used to achieve a basic result whereby only a single antigenic compound is presented to a given cell at a given point in time to thereby reduce or eliminate the immunodominance problem resulting from simultaneous presentation of more than one antigenic compound at the same time.

The invention also includes a method of making the particles and the population of groups of particles in order to form the formulation.

Formulations used to generate an immune response such as vaccine formulations of the invention can comprise polypeptides that correspond to an epitope, usually a T cell epitope, that, when brought into contact with a mammalian (e.g. human) immune system will elicit an immune response targeted to the corresponding epitope on a cell, virus, etc. Epitopes of interest include, without limitation, those present on pathogens, e.g. virus, bacteria, fungi, protozoan, etc.; and may further comprise epitopes associated with, for example, cancer cells. The polypeptides are encapsulated within and/or bound to the surface of specifically sized particles, where the particles may be formed of any suitable biocompatible material, e.g. biocompatible polymers such as poly(lactic-co-glycolic acid) (PLGA), polycaprolactone, polyglycolide, polylactic acid, poly-3-hydroxybutyrate, etc.

The invention provides optimally sized particles comprised of a biocompatible polymer, wherein the particles are only loaded with a population of identical compound species such as identical peptides, where the peptides may correspond to a single epitope, and further wherein the particles are sized so that only a single particle is taken into a single APC at a given point in time, and further wherein there are no other peptides or antigenic compounds in the particle.

An aspect of the invention is a composition comprising a first group of particles comprised of a biocompatible polymer and a first chemical species; and a second group of particles comprised of a biocompatible polymer and a second chemical species different from the first chemical species wherein each particle in the first group and the second group is substantially spherical, and has a diameter in a range from 10 microns±20% to 25 microns±20%; or 15 microns±20%.

In another aspect of the invention the first chemical species and second chemical species of the composition referenced above consist only of a first peptide and a second peptide consisting only of 8 to 20 amino acids; or 8 to 15 amino acids with no other antigenic compound present.

In yet another aspect of the invention the composition further comprises a third, and possibly further additional groups of particles (4, 5, 6 . . . 10 . . . 20 . . . 30, etc. groups) wherein each group is comprised of a biocompatible polymer and a chemical species different from the chemical species within the other groups of particles.

In another aspect of the invention the biocompatible polymer is a polymer selected of the group consisting of poly(lactic-co-glycolic acid) (PLGA), polycaprolactone, polyglycolide, polylactic acid, poly-3-hydroxybutyrate.

Another aspect of the invention is a use of a composition as described here in the manufacture of a formulation for treatment of a patient in order to generate an immune response within the patient.

Another aspect of the invention is a method of treatment which may be a method of generating an immune response in a subject by administering to a subject a formulation as described and disclosed herein and thereafter allowing the formulation to interact with the subject's immune system and thereby generate an immune response.

In another aspect of the invention the formulation is administered by injection.

In yet another aspect of the invention the formulation is administered by contacting the formulation with nasal membranes of the subject such as by the use of a nasal spray, nasal drops, or other formulation designed for administration to and contact with a subject's nasal membrane wherein the formulation may have a viscosity several times (2, 3, 4, etc) more viscous than water to reduce dripping of the formulation from the nasal cavity.

Another aspect of the invention is to provide a process whereby the particles as described above are formed by extrusion from a nozzle in a manner which creates particles and does not damage the antigenic compound, e.g. peptides, etc thereby allowing the component to substantially maintain its antigenic property.

Another aspect of the invention is to provide such a process for producing particles which can be carried out without the use of solvents including organic solvents or any other compounds beyond the biocompatible polymer and chemical species.

Also disclosed is a method of treatment, comprising:
administering to a subject a formulation comprising:
a first group of particles comprising a biocompatible polymer and consisting only of a first peptide species; and
a second group of particles comprising a biocompatible polymer and consisting only of a second peptide species different from the first peptide species; and
a third group of peptide particles comprising a biocompatible polymer and consisting only of a third peptide species different from both the first and second peptide species;
wherein the first, second, and third peptide species consists of 8 to 15 amino acids; and
allowing the groups of particles to interact with the subject's immune system and thereby generate an immune response to the first, second and third peptide species.

Another aspect on the invention is a method as recited above, wherein the formulation is administered by injection into the subject's lymph system.

Another aspect on the invention is a method as recited above, further comprising: administering an adjuvant to the subjects.

Another aspect on the invention is a method as recited above, wherein the amino acid sequences comprise epitopes of a pathogen or epitopes on the surface of a cell infected with a pathogen.

Another aspect on the invention is a method as recited above, wherein the pathogen is selected from the group consisting of a virus, a bacteria and a parasite.

Another aspect on the invention is a method as recited above, wherein the administering is by injecting the subject formulation with the formulation.

Another aspect on the invention is a method as recited above, wherein the administering is by contacting nasal membranes of the subject with the formulation which may be a viscous or sticky formulation designed for nasal administration, which includes a high viscosity carrier (5, 10 or more times the viscosity of water at 37° C.) or spheres coated with a bioadhesive.

An aspect of the invention is a vaccine formulation of the invention further comprising ink which would mark skin of a human patient as having been vaccinated.

Yet another aspect of the invention is an ink vaccine formulation wherein the ink is visible only under a black light.

Still another aspect of the invention is a vaccine formulation wherein the ink is encapsulated in microspheres wherein the microspheres are comprised of a biocompatible polymer and are 5 μm to 20 μm in diameter.

Still yet another aspect of the invention is a vaccine system whereby the ink in the vaccines is used to individually categorize the human patients vaccinated by imprinting a unique mark (e.g. number, letter, symbol or combination thereof) on each human patient vaccinated.

An aspect of the invention includes administering any formulation of the invention using a needle device conventionally used to draw a tattoo on a patient.

These and other objects, advantages, and features of the invention will become apparent to those persons skilled in the art upon reading the details of the formulations and methods of treatment as more fully described below.

### BRIEF DESCRIPTION OF THE DRAWINGS

FIG. 1 includes four photos (1A, 1B, 1C and 1D). Each of the photos show human dendrocytes and 10 micron diameter particles comprised of PLGA. In FIG. 1A two human dendrocytes are shown in proximity to the microspheres. In FIG. 1B a first human dendrocyte is shown phagocytosing three 10 micron PLGA microspheres with a second dendrocyte beginning phagocytosis. In FIG. 1C the first dendrocyte is shown completing phagocytosis of three 10 micron PLGA microspheres and a second dendrocyte in active phagocytosis. In FIG. 1D the second dendrocyte has completed phagocytosis of three 10 micron PLGA microspheres.

FIG. 2 includes four photos (2A, 2B, 2C and 2D) each of which show human dendrocytes and PLGA microspheres which are 15 microns in diameter. In FIG. 2A two human dendrocytes are in proximity with two 15 micron microspheres. In FIG. 2B a first dendrocyte is phagocytosing a 15 micron microsphere. In FIG. 2C a first dendrocyte has undergone complete phagocytosis of only a single 15 micron microsphere and a second dendrocyte is in active phagocytosis. In FIG. 2D a second dendrocyte has completed phagocytosis of a single 15 micron microsphere.

These still photos from FIGS. 1 and 2 were taken from videos of human dendrocytes placed in proximity to microspheres wherein the microspheres were 10 microns in diameter for FIGS. 1 and 15 microns in diameter for FIG. 2. The photos are part of many taken and were chosen from many other photos as photos which might best represent aspects of the invention.

FIG. 3 shows three images which schematically represent particles of the invention. In FIG. 3A the particles include a 1:1 ratio of a first antigen OVA to a second antigen VSV. FIG. 3B shows a schematic image of a particle of the invention wherein the ratio of the OVA antigen to the VSV antigen is 1:10. In FIG. 3C the ratio of the OVA antigen to the VSV antigen is 1:100.

FIG. 4 includes three sets of images. In FIG. 4A the images schematically represent a first group of particles which contain a first antigen OVA with a second image representing a second group of particles with a second antigen VSV wherein the first group of particles and second group of particles include a single antigen per particle. FIG. 4B includes two images wherein the first image represents a particle of the invention including a single antigen of OVA and the second image represents a particle which includes 10 antigens of VSV. FIG. 4C includes two images wherein the first image represents a group of particles wherein the particles include a single antigen of OVA and a second image which includes a group of particles wherein each particle includes 100 copies of the antigen VSV.

FIG. 5 shows a graph representing ratios of the data obtained using the particles in FIGS. 3 and 4. The graph of FIG. 5 is based on three data points wherein data point 1 is the ratio of the immune response obtained with particles of FIG. 3A relative to the immune response obtained with particles of FIG. 4A. Data point 2 is the ratio of the immune response obtained with particles of FIG. 3B relative to the immune response obtained with particles of FIG. 4B. Data point 3 is the ratio of the immune response obtained with particles of FIG. 3C relative to the immune response obtained with the particles of FIG. 4C. The results of FIG. 5 show that by separating antigens into two different groups of particles wherein the particles within each group include a different antigen from the other group a greater immune response is obtained.

FIG. 6 shows a plot and data related to Example 6 showing particle size distribution indicating highly uniform particles sizes.

### DETAILED DESCRIPTION OF THE INVENTION

Before the present composition, formulation and method of manufacture and use and treatment are described, it is to be understood that this invention is not limited to particular embodiment described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

A vaccine is a biological preparation intended to improve a recipient's immunity to a particular disease. A vaccine typically contains an agent that resembles a disease-causing microorganism, and is often made from weakened or killed forms of the microbe or its toxins. The agent stimulates the body's immune system to recognize the agent as foreign, destroy it, and “recognize” it, so that the immune system can more easily recognize and destroy any of these microorganisms that it later encounters. Vaccines can be prophylactic (e.g. to prevent or ameliorate the effects of a future infection by any natural or “wild” pathogen), or therapeutic (e.g. vaccines against cancer are also being investigated).

The expression “enhanced immune response” or similar term means that the immune response is elevated, improved or enhanced to the benefit of the host relative to the prior immune response status, for example, a native status before the administration of an immunogenic composition of the invention or to obtained a stronger immune response relative to the response in the absence of enhancement, e.g. 10%, 20% . . . 0.100% enhanced.

The terms “cell-mediated immunity” and “cell-mediated immune response” are meant to refer to the immunological defense provided by lymphocytes, such as that defense provided by T cell lymphocytes when they come into close proximity to a target cell. A cell-mediated immune response normally includes lymphocyte proliferation. When “lymphocyte proliferation” is measured, the ability of lymphocytes to proliferate in response to a specific antigen is measured. Lymphocyte proliferation is meant to refer to T-helper cell or cytotoxic T-lymphocyte (CTL) cell proliferation.

The term “immunogenic amount” refers to an amount of antigenic compound sufficient to stimulate an enhanced immune response, when administered with a subject immunogenic composition, as compared with the immune response elicited by the antigen in the absence of the microsphere formulation.

The terms “treatment”, “treating”, “treat” and the like are used herein to generally refer to obtaining a desired pharmacologic and/or physiologic effect such as an enhanced immune response. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers stimulating an immune response, and any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, e.g., arresting its development; or relieving the disease symptom, i.e., causing regression of the disease or symptom (c) reduction of a level of a product produced by the infectious agent of a disease (e.g., a toxin, an antigen, and the like); and (d) reducing an undesired physiological response to the infectious agent of a disease (e.g., fever, tissue edema, and the like).

The term “antigen presenting cell” or APC may generally refer to a mammalian cell having a surface HLA Class I or HLA Class II molecule in which an antigen is presented. Unless otherwise indicated, for the purposes of the present invention an antigen presenting cell is a “professional” antigen presenting cell that can activate or prime T cells, including naïve T cells. Professional APC usually express both HLA Class I and HLA Class II molecules, and are very efficient at internalizing antigens, either by phagocytosis or by receptor-mediated endocytosis, and then displaying an antigen or a fragment thereof bound to the appropriate HLA molecule on their cell surface. Synthesis of additional co-stimulatory molecules is a defining feature of professional APCs. Of these APCs, dendritic cells (DCs) have the broadest range of antigen presentation, and are the most important T cell activators. Macrophages, B cells and certain activated epithelial cells are also professional APCs.

### Invention in General

Formulations of the invention comprise one group or a plurality of groups of spherical particles, wherein the particles in each group are identical in terms of the antigen and further wherein the particles in different groups include a different active ingredient (antigen) from other groups. The formulation may be designed such that a population of antigen-presenting cells (APC) can be presented with a plurality of antigen species, formulated in such a way that any one antigen presenting cell will take up and present only a limited number of the antigen species, i.e. less than 5, less than 3, usually only a single antigen species. There is evidence that presentation of a plurality of epitopes by a single APC may result in immunodominance of a single epitope, which is undesirable in situations where overall responsiveness to the plurality of epitopes is desirable. For example, see Rodriguez, et al., “Immunodominance in Virus-Induced CD8+ T-Cell Responses Is Dramatically Modified by DNA Immunization and Is Regulated by Gamma Interferon” Journal of Virology, 76(9):4251-4259 (May 2002) and Yu et al., “Consistent Patterns in the Development and Immunodominance of Human Immunodeficiency Virus Type I (HIV-1)-Specific CD8+ T-Cell Responses following Acute HIV-1 Infection” Journal of Virology, 76(17):8690-9701 (September 2002), both incorporated herein by reference.

The invention accomplishes the desired result by placing an antigen, for example a peptide antigen (or plurality of identical peptide antigens) as defined herein, on or in a particle of a defined size of a biocompatible polymer, usually in a form that is approved by the United States Food and Drug Administration for administration to humans. Vaccine formulations may be comprised of a pharmaceutically acceptable carrier. The carrier can come in a variety of forms depending on the mode of administration such as injection, nasal, inhalation, oral, etc. In addition to the carrier the formulation includes a plurality of spherical particles which particles are comprised of a biocompatible polymer and an antigen, e.g. a peptide antigen. The particles may be generally spherical in shape and have a defined diameter in a range of 10 microns to 20 microns, or about 10 microns±20%, or ±10% or ±5% or ±2%. The diameter of the particles is designed such that an antigen presenting cells such as dendritic cell can consume only a single particle, and not consume two or more particles. Each particle may contain a large number of identical compounds, for example peptide species, although the antigen species within any given particle is identical. Thus, each group of particles consist essentially only of copies of one compound, e.g. peptides which have the same amino acid sequence. In some circumstances antigen containing particles may be allowed to include more than a single compound or species, e.g. two, three, four or five compounds, provided that, in the case of peptides, the different compounds included do not exhibit immunodominance with respect to each other.

Compositions or formulations of the invention are comprised of a plurality of groups of particles. There may be a plurality of groups which may comprise two or more groups, three or more groups, five or more groups, ten or more groups, twenty or more groups, twenty five or more groups, fifty or more groups, or any desired number of groups. Any group of particles may include a plurality of particles meaning that it may include two or more, three or more, five or more, ten or more, twenty or more, twenty five or more, fifty or more, a hundred or more, a thousand or more particles within any particular group. The particles from a single group consists essentially only of a single antigen species to avoid immunodomination. Alternatively the particles from a single group may consist essentially of a single antigen species that is presented by a class I protein of the APC. Further, the number of particles within one group may be the same as or may be different from the number of particles within another group.

Some examples of biocompatible polymers useful in the present invention include hydroxyaliphatic carboxylic acids, either homo- or copolymers, such as poly(lactic acid), poly(glycolic acid), Poly(dl-lactide/glycolide, poly(ethylene glycol); polysaccharides, e.g. lectins, glycosaminoglycans, e.g. chitosan; celluloses, acrylate polymers, and the like. The particle size is selected to (a) be sufficiently small that it is capable of uptake and processing by an antigen presenting cell; and (b) be sufficiently large that an APC will generally not be capable of taking up more than one particle.

Each set, or group, of particles comprises a single antigen, e.g. peptide species, e.g. peptides of identical sequences. The peptide antigen may be other than a sequence determined to be an immunodominant sequence.

Groups of particles may be combined as a composition or formulation comprising particles with a plurality of peptide species. For example, two groups of particles provide at least two peptide species and larger numbers of groups can provide, at least 3, at least 4, at least 5, at least 10, at least 20, and usually not more than 50, not more than 40, not more than 30 peptide species via corresponding groups of spheres. In one embodiment, all of the particles are substantially spherical and have a diameter in a range of more than 10 microns and less than 20 microns. Each particle is comprised of a biocompatible polymer and with respect to peptides consists only of a single species of peptide, but include a plurality of copies of that single species.

In some embodiments the antigen is embedded in the particle, for example by mixing peptides and polymers prior to formation of the particles. In other embodiments the antigens are coupled to the particle surface or the particles are encapsulated inside an outer spherical shell. The spherical particle surface may be optionally textured to simulate, to a degree, the surface of an infectious bacteria, virus or other pathogen.

Particles in a formulation may be heterogenous or homogenous in size, usually homogeneous, where the variability may be not more than 100% of the diameter, not more 50%, not more than 20%, not more than 10%, not more than 2%, etc. Particle sizes are may be about 8 μm in diameter, about 10 μm, about 12 μm about 14 μm, about 15 μm, about 16 μm, about 17, about 18 μm, about 20 μm, not more than about 25 μm diameter.

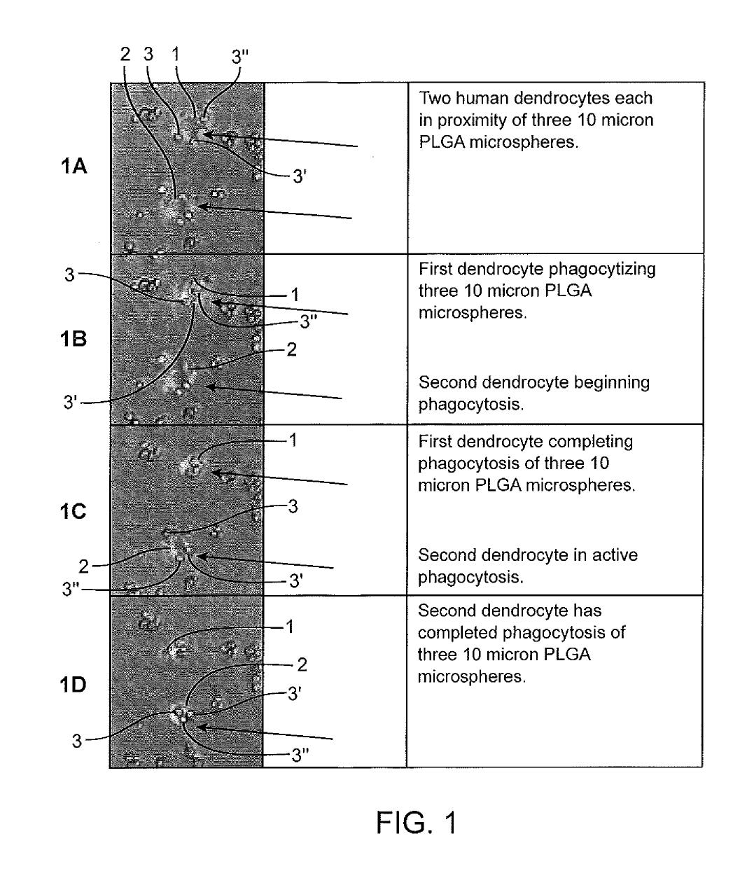

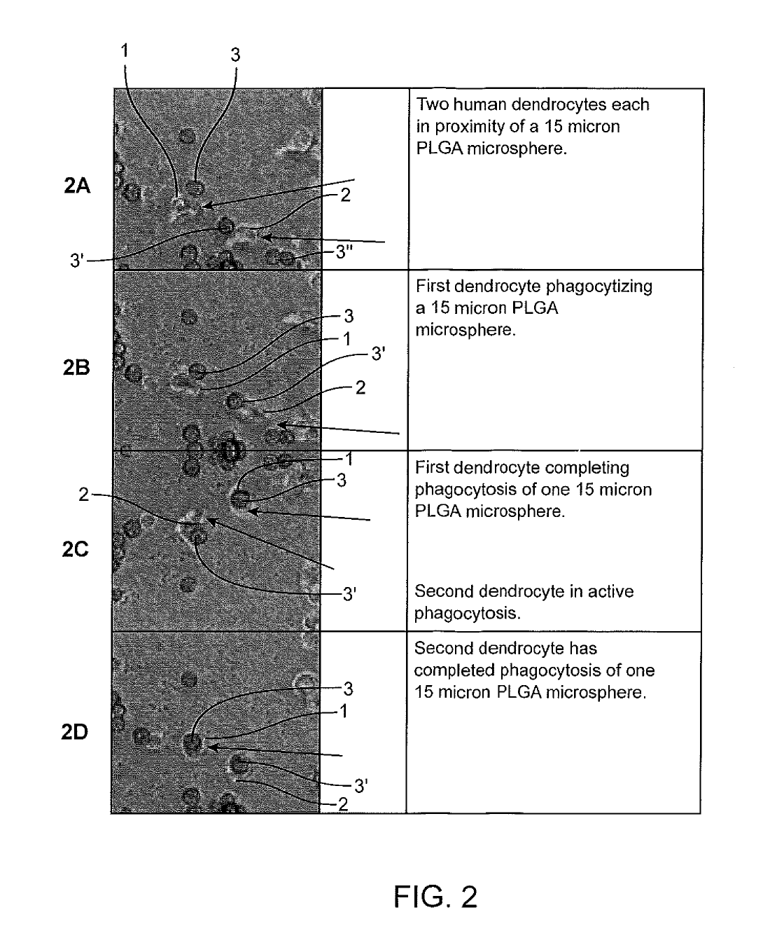

The optimum size for a particular peptide or class of peptides may be determined empirically by various methods. For example, two different peptides may be detectably labeled with two different fluorophores, and used to prepare particles of the invention. A mixture of the particles is provided to antigen presenting cells, which are then viewed by optical microscope, flow cytometry, etc. to determine if a single fluorophore or if multiple fluorophores are present in any single APC, where the size of particle that provides for exclusive uptake is chosen (see FIGS. 1 and 2). Functional tests may also be performed, e.g. by providing particles with the cognate antigens for different T cell lines and determining if one or both lines are activated by an APC.

In order to determine the precise size which is desirable for the particles, various types of labeling can be used. In addition to the fluorophores referred to above, labeling can be performed with semiconductor nanocrystals which are generally referred to as quantum dots. The purpose of carrying out the experiment is to determine a size at which the antigen presenting cells such as the macrophage can consume only a single particle. The size would be too large if the macrophage cannot consume the particle. The size would be too small if the macrophage can consume more than one particle.

The optimum size of particle to achieve the desired result may vary depending on the charge of the peptide that is being presented, for example a positively charged peptide may be more readily ingested by an APC than a neutral or negatively charged peptide. In some embodiments each peptide is individually optimized for a microsphere size that achieves exclusive uptake, and thus a formulation of a plurality of microsphere/peptide combinations may be heterogenous in size, although the size for a peptide species will be narrowly defined.

Based on the above it will be understood that one aspect of the invention is the use of groups of particles of the invention to determine information about the operation of the immune system, particularly with respect to antigen presentation. The groups of particles provide information on how the immune system deals with different antigens in the particles. Thus, the groups of particles of the invention can provide valuable information on how an immune system operates and as such what formulations will provide a desired immune response.

In some embodiments of the invention, a formulation is provided of PLGA microspheres of a defined size from 14 μm to 16 μm in diameter (±20%, ±10% m±5%, ±2%), where each microsphere comprises a single peptide antigen species, and where from 10 to 30 different peptide antigen species are present in the formulation via 10 to 30 different groups of particles. In some embodiments the peptide antigen is an antigen of a virus such as hepatitis C virus (HCV) or human immunodeficiency virus (HIV-1).

### Antigen Presenting Cells

The three major classes of antigen presenting cells are dendritic cells (DCs), macrophages, and B cells, but dendritic cells are considerably more potent on a cell-to-cell basis and are the only antigen presenting cells that activate naïve T cells. DC precursors migrate from bone marrow and circulate in the blood to specific sites in the body, where they mature. This trafficking is directed by expression of chemokine receptors and adhesion molecules. Upon exposure to antigen and activation signals, the DCs are activated, and leave tissues to migrate via the afferent lymphatics to the T cell rich paracortex of the draining lymph nodes. The activated DCs then secrete chemokines and cytokines involved in T cell homing and activation, and present processed antigen to T cells. The groups of particles of the invention provide information on how to best present processed antigens to T cells to obtain a desired immune response.

DCs mature by upregulating costimulatory molecules (CD40, CD80 and CD86), and migrate to T cell areas of organized lymphoid tissues where they activate naive T cells and induce effector immune responses. In the absence of such inflammatory or infectious signals, however, DCs present self-antigens in secondary lymphoid tissues for the induction and maintenance of self-tolerance. Dendritic cells include myeloid dendritic cells and plasmacytoid dendritic cells.

For purposes of the invention, e.g. determining the uptake of particles of any formulation including a vaccine formulations by APC, any one of the classes of APC may be used, including without limitation immature DC, monocytes, mature myeloid DC, mature pDC, etc. For example see Foged et al (2005) International Journal of Pharmaceutics 298(2): 315-322; Reece et al. (2001) Immunology and Cell Biology 79:255-263; Tel et al. (2010) J. Immunol. 184:4276-4283, each herein specifically incorporated by reference.

### Antigens

The term “antigen” as used herein includes meanings known in the art, and means a molecule or portion of a molecule, frequently for the purposes of the present invention a polypeptide molecule (amino acid sequence), that can react with a recognition site on an antibody or T cell receptor. The term “antigen” also includes a molecule or a portion of a molecule that can, either by itself or in conjunction with an adjuvant or carrier, elicit an immune response (also called an “immunogen”). The groups of particles of the invention provide information on how to best structure antigen components in the particles to increase or decrease an immune response in a desired way.

The “specificity” of an antibody or T cell receptor refers to the ability of the variable region to bind with high affinity to an antigen. The portion of the antigen bound by the immune receptor is referred to as an epitope, and an epitope is that portion of the antigen which is sufficient for high affinity binding. An individual antigen typically contains multiple epitopes, although there are instances in which an antigen contains a single epitope. In some embodiments of the invention, a plurality of peptide fragments representing individual epitopes are derived from a protein antigen. Where the antigen is a peptide, generally a linear epitope will be at least about 8 amino acids in length, and not more than about 15 to 22 amino acids in length. A T cell receptor recognizes a more complex structure than antibodies, and requires both a major histocompatibility antigen binding pocket and an antigenic peptide to be present. The binding affinity of T cell receptors to epitopes is lower than that of antibodies to epitopes, and will usually be at least about 10⁻⁴ M, more usually at least about 10⁻⁶ M, 10⁻⁶M, 10⁻⁷M.

Antigens of interest for the purposes of the invention include pathogens and portions thereof, e.g. proteins, peptides, polysaccharides, etc., e.g. virus, bacteria, protozoans, etc.; tumor antigens, and the like. Viral pathogens of interest include retroviral pathogens, e.g. HIV-1; HIV-2, HTLV, FIV, SIV, etc.; influenza, smallpox (vaccinia), measles, mumps, rubella, poliovirus, rotavirus, varicella (chickenpox), hepatitis A, B, C, D virus, bacterial antigens, tumor antigens, and the like. Microbes of interest include, but are not limited to the following, include: Citrobacter sp.; Enterobacter sp.; Escherichia sp., e.g. E. coli; Klebsiella sp.; Morganella sp.; Proteus sp.; Providencia sp.; Salmonella sp., e.g. S. typhi, S. typhimurium; Serratia sp.; Shigella sp.; Pseudomonas sp., e.g. P. aeruginosa; Yersinia sp., e.g. Y. pestis, Y. pseudotuberculosis, Y enterocolitica; Franciscella sp.; Pasturella sp.; Vibrio sp., e.g. V. cholerae, V. parahemolyticus; Campylobacter sp., e.g. C. jejuni; Haemophilus sp., e.g. H. influenzae, H. ducreyi; Bordetella sp., e.g. B. pertussis, B. bronchiseptica, B. parapertussis; Brucella sp., Neisseria sp., e.g. N. gonorrhoeae, N. meningitidis, etc. Other bacteria of interest include Legionella sp., e.g. L. pneumophila; Listeria sp., e.g. L. monocytogenes; Staphylococcus sp., e.g. S. aureus Mycoplasma sp., e.g. M. hominis, M. pneumoniae; Mycobacterium sp., e.g. M. tuberculosis, M. leprae; Treponema sp., e.g. T. pallidum; Borrelia sp., e.g. B. burgdorferi; Leptospirae sp.; Rickettsia sp., e.g. R. rickettsii, R. typhi; Chlamydia sp., e.g. C. trachomatis, C. pneumoniae, C. psittaci; Helicobacter sp., e.g. H. pylori, etc.

Antigenic peptides can include purified native peptides, synthetic peptides, recombinant proteins, crude protein extracts, attenuated or inactivated viruses, cells, micro-organisms, or fragments of such peptides. Immunogenic peptides can be native or synthesized chemically or enzymatically. Any method of chemical synthesis known in the art is suitable. Solution phase peptide synthesis can be used to construct peptides of moderate size or, for the chemical construction of peptides, solid phase synthesis can be employed. Atherton et al. (1981) Hoppe Seylers Z. Physiol. Chem. 362:833-839. Proteolytic enzymes can also be utilized to couple amino acids to produce peptides. Kullmann (1987) Enzymatic Peptide Synthesis, CRC Press, Inc. Alternatively, the peptide can be obtained by using the biochemical machinery of a cell, or by isolation from a biological source. Recombinant DNA techniques can be employed for the production of peptides. Hames et al. (1987) Transcription and Translation: A Practical Approach, IRL Press. Peptides can also be isolated using standard techniques such as affinity chromatography.

The groups of particles of the invention can be used to determine which antigens and antigen synthesis techniques result in obtaining an immune response desired.

### Type of Vaccines

The groups of particles of the invention can use epitope portions of vaccine materials as described below to determine which positions of these materials obtain the desired immune response. Such information can be used to create safer, less expensive and more effective vaccines.

Some vaccines contain inactivated, but previously virulent, micro-organisms that have been destroyed, i.e. rendered inactive such as with chemicals or heat. Examples are the influenza vaccine, cholera vaccine, bubonic plague vaccine, polio vaccine, hepatitis A vaccine, and rabies vaccine. Other vaccines contain live, attenuated microorganisms. Many of these are live viruses that have been cultivated under conditions that disable their virulent properties, or which use closely-related but less dangerous organisms to produce a broad immune response, however some are bacterial in nature. They typically provoke more durable immunological responses and are the preferred type for healthy adults. Examples include the viral diseases yellow fever, measles, rubella, and mumps and the bacterial disease typhoid. The live Mycobacterium tuberculosis vaccine developed by Calmette and Guérin is not made of a contagious strain, but contains a related species called “BCG”.

Toxoid vaccines are made from inactivated toxic compounds that cause illness rather than the micro-organism. Examples of toxoid-based vaccines include tetanus and diphtheria. Toxoid vaccines are known for their efficacy. Not all toxoids are for micro-organisms; for example, Crotalus atrox toxoid is used to vaccinate dogs against rattlesnake bites.

In subunit vaccine, a fragment of a microorganism is utilized to induce an immune response. Examples include the subunit vaccine against Hepatitis B virus, which is composed of only the surface proteins of the virus (previously extracted from the blood serum of chronically infected patients, but now produced by recombination of the viral genes into yeast); the virus-like particle (VLP) vaccine against human papillomavirus (HPV) that is composed of the viral major capsid protein, and the hemagglutinin and neuraminidase subunits of the influenza virus.

In conjugate vaccines, the polysaccharides present on bacterial outer coats are conjugated to a carrier to induce a more robust response, e.g. in the Haemophilus influenzae type B vaccine.

Vaccines currently in development include dendritic cell vaccines, which combine dendritic cells with antigens, which have shown some positive preliminary results for treating brain tumors. In recombinant vector vaccines, the physiology of one micro-organism is combined with the DNA of the other, to create immunity against diseases that have complex infection processes. DNA vaccination, created from an infectious agent's DNA, has been developed. It works by insertion (and expression, triggering immune system recognition) of viral or bacterial DNA into human or animal cells. Some cells of the immune system that recognize the proteins expressed will mount an attack against these proteins and cells expressing them. Because these cells live for a very long time, if the pathogen that normally expresses these proteins is encountered at a later time, they will be attacked instantly by the immune system. One advantage of DNA vaccines is that they are very easy to produce and store. T-cell receptor peptide vaccines are under development for several diseases using models of Valley Fever, stomatitis, and atopic dermatitis. These peptides have been shown to modulate cytokine production and improve cell mediated immunity. Other vaccines are in development to target bacterial proteins that are involved in complement inhibition, which would neutralize a key bacterial virulence mechanism.

While most vaccines are created using inactivated or attenuated compounds from micro-organisms, synthetic vaccines are composed mainly or wholly of synthetic peptides, carbohydrates or antigens.

Vaccines may be monovalent (also called univalent) or multivalent (also called polyvalent). A monovalent vaccine is designed to immunize against a single antigen or single microorganism. A multivalent or polyvalent vaccine is designed to immunize against two or more strains of the same microorganism, or against two or more microorganisms. In certain cases a monovalent vaccine may be preferable for rapidly developing a strong immune response.

In order to provide the best protection against an infection, children are recommended to receive vaccinations as soon as their immune systems are sufficiently developed to respond to particular vaccines, with additional “booster” shots often required to achieve “full immunity”. This has led to the development of complex vaccination schedules. In the United States, the Advisory Committee on Immunization Practices, which recommends schedule additions for the Centers for Disease Control and Prevention, recommends routine vaccination of children against: hepatitis A, hepatitis B, polio, mumps, measles, rubella, diphtheria, pertussis, tetanus, HiB, chickenpox, rotavirus, influenza, meningococcal disease and pneumonia. The large number of vaccines and boosters recommended (up to 24 injections by age two) has led to problems with achieving full compliance. In order to combat declining compliance rates, various notification systems have been instituted and a number of combination injections are now marketed (e.g., Pneumococcal conjugate vaccine and MMRV vaccine), which provide protection against multiple diseases.

Besides recommendations for infant vaccinations and boosters, many specific vaccines are recommended at other ages or for repeated injections throughout life—most commonly for measles, tetanus, influenza, and pneumonia. Pregnant women are often screened for continued resistance to rubella. The human papillomavirus vaccine is currently recommended in the U.S. and UK for ages 11-25. Vaccine recommendations for the elderly concentrate on pneumonia and influenza, which are more deadly to that group. In 2006, a vaccine was introduced against shingles, a disease caused by the chickenpox virus, which usually affects the elderly.

Use of groups of particles of the invention can provide information that will allow for the reformulation of current vaccines, e.g. reducing undesired immune responses via small antigen components, improving patient compliance by obtaining an immune response that does not require a follow-up booster. Information obtained via the groups of particles of the invention can also be combined with patient specific HLA information to tailor immunogens to specific groups of HLA types.

### Tattoo Ink

A vaccine formulation of the invention may comprise ink to mark the skin of the human patient to which the vaccine is administered. The ink may be conventional ink used in tattooing, or may be ink that is only visible under a specific type of light such as laser light, or a black light that emits ultraviolet radiation. The ink and vaccine may be administered in a single formulation or in two separate formulations. The ink may be free in the formulation or may be encapsulated in microparticles of similar size to vaccine particles of the invention. The ink may be encapsulated by a process as disclosed within U.S. Pat. No. 6,116,516 issued Sep. 12, 2000 to Alfonso Ganan-Calvo, herein incorporated by reference. The ink particles may be smaller than any vaccine particle because the immunodominance problem is not an issue with ink. The small encapsulate ink particles may enhance the immune response to be generated in response to the antigens of the formulation. Further, enhancement of that immune response may be generated by using a tattoo needle administering device to administer the formulation.

### General Overview

Vaccine compositions of the invention may be formulated for delivery to a patient via a tattoo needle or similar device. Such vaccine compositions may include a tattoo ink that can be used to create an identifying marking on a patient for purposes of, e.g., identifying the person, the date of administration and/or the particular vaccine composition that was administered to the patient. In some embodiments, tattoo ink compositions are provided wherein the tattoo ink does not comprise a vaccine, and is simply used to create an identifying marking on the patient's skin.

Suitable tattoo inks for use with the compositions and methods of the present disclosure include tattoo inks that are visible to the naked eye, as well as tattoo inks that are invisible to the naked eye, but which may be selectively activated for identification purposes. Other suitable tattoo inks include erasable or removable tattoo inks that can be removed when no longer needed, and tattoo ink compositions that are not permanent, such as those that are designed to fade away and disappear with time. Exemplary tattoo ink compositions are provided below.

### Traditional Tattoo Ink Compositions

In some embodiments of the present disclosure, tattoo ink compositions may comprise traditional tattoo pigments and dyes, such as, e.g., India ink. Traditional tattoo inks are generally visible with the naked eye and are commercially available from a variety of sources.

### Semi-Permanent Tattoo Ink Compositions

In some embodiments of the present disclosure, tattoo ink compositions are provided which remain in the dermis for a predetermined period of time (e.g., 3, 6, 9, months or 1, 2, 5, 10 years, etc.) and then spontaneously disappear. These “semi-permanent” or “temporary” tattoo inks are produced by entrapping, encasing, complexing, incorporating, or encapsulating appropriate pigments (pigments which are readily eliminated when present by themselves in the dermis) into tattoo pigment vehicles which slowly bioabsorb, bioerode, or biodegrade over a predetermined period of time, such as at a constant rate slowly over a five-year period, or can release the pigments over a short period of time once a specific percentage of the tattoo pigment vehicle has been absorbed. For example, all of the pigment may be released between the fourth and fifth years.

One example of a tattoo pigment vehicle which releases the pigment continuously over a predetermined period is one in which the pigment is incorporated or mixed in throughout the entire substance of the tattoo pigment vehicle to form color-carrying particles. When these pigment/vehicle complexes are introduced into the dermis (in the form of a tattoo), the tattoo pigment vehicle slowly bioabsorbs, releasing the pigment from the dissolving tattoo pigment vehicle maturely, eliminating the pigment from the dermis. When all of the pigment/vehicle complexes have been absorbed, the tattoo is no longer visible.

To release the pigment over a short period of time, bioabsorbable microcapsules or microfiakes are used as the tattoo pigment vehicle. With microcapsules, pigment/vehicle complexes comprise a core of pigment surrounded by the tattoo pigment vehicle, which maintains its integrity until a certain threshold percentage of the tattoo pigment vehicle is dissolved, bioeroded, or bioabsorbed. At this point, the tattoo pigment vehicle no longer protects the pigment from elimination. The pigment is released into the dermis, where it is eliminated over a relatively short period of time.

Alternatively, microfiakes made of pigment and tattoo pigment vehicle, in which the pigment is mixed throughout the microfiakes, maintain a relatively consistent pigmented surface area during the process of bioabsorption. Over a predetermined period of time, the visible pigmented surface dissolves, similar to the melting of a frozen lake or pond.

The tattoo pigment vehicle for the pigment or dye comprises any biologically tolerated material that retains the pigment or dye in the dermis, for whatever time or under whatever conditions are desired. In any of these cases, the tattoo pigment vehicle carries a colored pigment or dye which can be administered into the dermis in any pattern or configuration in a manner similar to conventional tattooing. The tattoo pigment vehicle is sufficiently transparent or translucent so as to permit the color of the pigment or dye to show through and be visible. Preferably, the pigment or dye comprises particles smaller than 1 micron.

For producing semi-permanent tattoos, the pigments or dyes are entrapped, encased, complexed, incorporated, encapsulated, or otherwise associated in or with tattoo pigment vehicles composed of bioabsorbable, bioerodible, or biodegradable material. The material is designed to bioabsorb, bioerode, or biodegrade over a predetermined period of time so that the tattoo ink, when administered into the dermis, creates a tattoo which lasts only until the tattoo pigment vehicle bioabsorbs. Upon partial or complete bioabsorption of the tattoo pigment vehicle, the pigment or dye is released, allowing its elimination from the dermis.

A great many biodegradable polymers exist, and the length of time which the tattoo lasts in a visible state in the dermis is determined by controlling the type of material and composition of the tattoo pigment vehicle. Among the bioabsorbable, bioerodible, or biodegradable polymers that can be used are those disclosed in Higuchi et al., U.S. Pat. Nos. 3,981,303, 3,986,510, and 3,995,635, including zinc alginate poly(lactic acid), poly(vinyl alcohol), polyanhydrides, and poly(glycolic acid). Alternatively, microporous polymers are suitable, including those disclosed in Wong, U.S. Pat. No. 4,853,224, such as polyesters and polyethers, and Kaufman, U.S. Pat. Nos. 4,765,846 and 4,882,150.

Other polymers which degrade slowly in vivo are disclosed in Davis et al., U.S. Pat. No. 5,384,333, which are biodegradable polymers which are solid at 20-37° C. and are flowable, e.g., a liquid, in the temperature range of 38-52° C. In preparing a semi-permanent tattoo, the dye or pigment is incorporated in the polymer matrix, and the system can be warmed to approximately 50° C., where it liquefies. The system is then injected into the dermis in a desired tattoo design, where it cools and re-solidifies.

For this type of semi-permanent tattoo pigment vehicle, any biodegradable polymer system which has the following characteristics can be used, including homopolymers, copolymers, block copolymers, waxes and gels, as well as mixtures thereof. A preferred polymer system is a triblock copolymer of the general formula: A-B-A, where A represents a hydrophobic polymer block, and B represents a hydrophilic polymer. The monomers and polymers are preferably linked through ester groups. Preferred hydrophobic polymers and oligomers include, but are not limited to, units selected from polyglycolic acid, polyethylene terephthalate, polybutyl lactone, polycaprolactone, D-polylactic acid, polytetrafluoroethylene, polyolefins, polyethylene oxide, polylactic acid, polyglutamic acid, polyL-lysine, and poly-L-aspartic acid. Preferred hydrophilic polymers include polyethylene glycol, polypropylene glycol, and poly(vinyl alcohol).

Hydrogel matrices or tattoo pigment vehicles for preparing semi-permanent tattooing inks are formed by crosslinking a polysaccharide or a mucopolysaccharide with a protein and loading the dye or pigment into the hydrogel matrices. Proteins include both full-length proteins and polypeptide fragments, which in either case may be native, recombinantly produced, or chemically synthesized. Polysaccharides include both polysaccharides and mucopolysaccharides.

A hydrogel in which the pigment or dye can be incorporated into a tattoo ink is disclosed in Feijen, U.S. Pat. No. 5,041,292. This hydrogel comprises a protein, a polysaccharide, and a cross-linking agent providing network linkages therebetween wherein the weight ratio of polysaccharide to protein in the matrix is in the range of about 10:90 to about 90:10. The pigment or dye is mixed into this matrix in an amount sufficient to provide color when the hydrogel matrix is administered to the dermis.

Examples of suitable polysaccharides include heparin, fractionated heparins, heparan, heparan sulfate, chondroitin sulfate, and dextran, including compounds described in U.S. Pat. No. 4,060,081 to Yannas et al. Using heparin or heparin analogs results in reduced immunogenicity in terms of a response to the heparin. The protein component of the hydrogel may be either a full-length protein or a polypeptide fragment. The protein may be in native form, recombinantly produced, or chemically synthesized. The protein composition may also be a mixture of full-length proteins and/or fragments. Typically, the protein is selected from the group consisting of albumin, casein, fibrinogen, gamma-globulin, hemoglobin, ferritin and elastin. The protein component may also be a synthetic polypeptide, such as poly(a-amino acid), polyaspartic acid or polyglutamic acid. Albumin is the preferred protein component of the matrix, as it is an endogenous material which is biodegradable in blood and tissue by proteolytic enzymes. Furthermore, albumin prevents adhesion of thrombocytes and is nontoxic and nonpyrogenic.

In forming hydrogels containing pigments or dyes; the polysaccharide or mucopolysaccharide and the protein are dissolved in an aqueous medium, followed by addition of an amide bond-forming cross-linking agent. A preferred crosslinking agent for this process is a carbodiimide, preferably the water-soluble diimide N-(3-dimethyl-aminopropyl)-N-ethylcarbodiimide. In this method, the cross-linking agent is added to an aqueous solution of the polysaccharide and protein at an acidic pH and a temperature of about 0 to 50° C., preferably from about 4 to about 37° C., and allowed to react for up to about 48 hours. The hydrogel so formed is then isolated, typically by centrifugation, and washed with a suitable solvent to remove uncoupled material.

Alternatively, a mixture of the selected polysaccharide or mucopolysaccharide and protein is treated with a crosslinking agent having at least two aldehyde groups to form Schiff-base bonds between the components. These bonds are then reduced with an appropriate reducing agent to give stable carbon-nitrogen bonds.

Once the hydrogel is formed, it is loaded with the pigment or dye by immersing the hydrogel in a solution or dispersion of the pigments or dye. The solvent is then evaporated. After equilibration, the loaded hydrogels are dried in vacuo under ambient conditions and stored.

Virtually any pigment or dye may be loaded into the hydrogel vehicles, providing that surface considerations, such as surface charge, size, geometry and hydrophilicity, are taken into account. For example, incorporation and release of a high-molecular weight dye will typically require a hydrogel having a generally lower degree of cross-linking. The release of a charged pigment or dye will be strongly influenced by the charge and charge density available in the hydrogel, as well as by the ionic strength of the surrounding media.

The rate of pigment or dye release from the vehicles can also be influenced by post-treatment of the hydrogel formulations. For example, heparin concentration at the hydrogel surface can be increased by reaction of the formulated hydrogels with activated heparin (i.e., heparin reacted with carbonyldiimidazole and saccharine) or with heparin containing one aldehyde group per molecule. A high concentration of heparin at the hydrogel surface will form an extra “barrier” for positively charged dyes or pigments at physiological pH values. Another way of accomplishing the same result is to treat the hydrogels with positively charged macromolecular compounds like protamine sulfate, polylysine, or like polymers. Another way of varying hydrogel permeability is to treat the surfaces with biodegradable block copolymers containing both hydrophilic and hydrophobic blocks. The hydrophilic block can be a positively charged polymer, like polylysine, while the hydrophilic block can be a biodegradable poly(a-amino acid), such as poly(L-alanine), poly(L-leucine), or similar polymers.

Another slow-release system used as a tattoo pigment vehicle for pigments or dyes to form a semi-permanent tattoo is a dye or pigment and an enzyme encapsulated within a microcapsule having a core formed of a polymer which is specifically degraded by the enzyme and a rate controlling skin. The integrity of the shell is lost when the core is degraded, causing a sudden release of pigment or dye from the capsule. In this type of system, the microcapsule consists of a core made up of a polymer around which there is an ionically-bound skin or shell. The integrity of the skin or shell depends on the structure of the core. An enzyme is encapsulated with the biologically-active substance to be released during manufacture of the core of the microcapsule. The enzyme is selected to degrade the core to a point at which the core can no longer maintain the integrity of the skin, so that the capsule falls apart. An example of such as system consists of an ionically cross-linked polysaccharide, calcium alginate, which is ionically coated with a polycationic skin of poly-L-lysine. The enzyme used to degrade the calcium-alginate coated with poly-L-lysine microcapsules is an alginase from the bacteria Beneckea pelagio or Pseudomonas putida. Enzymes exist that degrade most naturally-occurring polymers. For example, the capsule core may be formed of chitin for degradation with chitinase.

Other natural or synthetic polymers may also be used and degraded with the appropriate enzyme, usually a hydrogenase.

A particularly preferred bioabsorbable polymer vehicle is a triblock copolymer of poly caprolactone-polyethylene glycol-poly caprolactone. This polymer contains ester bonds which hydrolyze in a hydrophilic environment. The biodegradable polymer matrix should comprise about 30-99% of the tattoo ink.

Several mechanisms are involved in the rate and extent of dye or pigment release. In the case of very high molecular weight pigments, the rate of release is more depending on the rate of tattoo pigment vehicle bioabsorption. With lower molecular weight pigments, the rate of pigment release is more dominated by diffusion. In either case, depending on the tattoo pigment vehicle composition selected, ionic exchange can also play a major role in the overall release profile.

### Selectively Activatable Tattoo Ink Compositions (e.g., pH Sensitive)

Formulations of the invention may comprise a selectively activatable tattoo ink composition and system, wherein the tattoo ink is changed in color and even appears to disappear by becoming colorless, with the process being reversible and repeatable. The compositions and systems disclosed herein are particularly well suited for use with a pH-sensitive tattoo ink composition, and will be described below. The compositions and systems of the present disclosure are equally well suited for use with other types of chemical sensitivity, such as salinity, glucose levels, etc.

In one embodiment, the present invention provides a type of tattoo ink that is visible or invisible under ambient light until an activating chemical is applied to it. This chemically reactive adjustable tattoo ink creates a reversible chemical change in the ink which results in the ink changing color, becoming visible or invisible. The tattoo is formed by injection of the selectively alterable intermittent tattoo ink (reactive pigment), along with an inert carrier and a temporary activating agent (so the ink will at least be temporarily visible during application), into the skin with a traditional tattoo delivery system. The selectively alterable intermittent tattoo ink can then be altered (invisible ink activated, visible ink deactivated, etc.) such as by the application of an activating agent either to the skin over the tattoo site or by a normal or abnormal physiological chemical change within the body. The activating agent is removed from the skin by washing, by the application of a neutralizing agent, or by the natural metabolic functions of the body resulting in the tattoo being invisible or being returned to its deactivated state.

The selectively alterable intermittent tattoo ink of the present disclosure may be applied as any other tattoo ink, and will provide the wearer with a permanent tattoo that will be visible only if and when the wearer wishes, and the colors of which are adjustable and reversible.

In one embodiment, the tattoo ink system comprises pH sensitive pigments, a harmless non-pigmented carrier, an activating agent (such as an ointment which may be applied with a pre-moistened disposable towelette wipe, disclosed in U.S. Pat. No. 6,287,582, or a normal or abnormal physiological change), and a neutralizing agent (such as an ointment, spray-on mist, or pre-moistened disposable towelette wipe). Both the activating agent and neutralizing agent can be applied with a towelette, spray-on mist, or ointment. The pigment in the ink can be activated in a number of ways; by application of a solution of specific pH value such as by way of a pre-moistened wipe, spray-on mist, or application of an ointment of specific pH to the tattoo location in which the H+ ions will diffuse into or out of the skin by way of osmosis activating the pigments; by a normal physiological change in pH created by heavy exercise as in exercise-induced acidosis; or by a pathological acidosis such as metabolic acidosis.

Non-limiting examples of suitable pH-sensitive pigments include: bromocresol purple, chlorophenol red, bromophenol blue, p-nitrophenol, phenol red, neutral red, rosolic red, cresol red, thymol blue, and phenolpthalein. These chemicals are reagents that, when exposed to changing pH levels, react by changing color. By combining the above chemicals, tattoo ink compositions can be created that are invisible, change color, change shape, change size, or disappear when exposed to the appropriate pH.

While only blood pH values ranging from 6.8 to 7.8 are compatible with life, the physiological pH of the skin typically ranges from 5.0 to 9.0. Commercial skin products are known and available on the market boasting as being noncorrosive even at pH levels as low as 1.0 (the exemplary pigments listed above all react within a pH range from 5.0 to 9.0.) In general, the compositions of the present disclosure have pigments that are capable of reacting over a pH value of between 4.4 and 10.5.

By preparing a tattoo ink which utilizes a pH sensitive pigment, such as those previously described, or the indicators listed in Table 1, a chemically adjustable intermittent tattoo ink can be made, wherein the tattoo ink changes in color such that the tattoo can change in appearance, color and even vanish. This color change process is fully reversible.

A suitable carrier of the pH sensitive pigment chemicals can be glycerin, ethyl alcohol, purified water, witch hazel, as well as any other existing carriers that are in use in the tattoo industry and are known to those skilled in the art. The tattoo ink is generally prepared at a pH level necessary for it to be seen in its activated color so it may be accurately drawn with. The buffering system of the human body brings the pH of the injection site (tattoo needle sight) to within the normal physiological intracellular range of pH from 6.8 to 7.4 at which time the tattoo would change to its deactivated state.

One embodiment of an activating agent for the tattoo ink of the present disclosure is made with Beckman Hydroxide pH Buffer at pH 12.45 and distilled water. Other embodiments include: sodium phosphate dibasic, sodium hydroxide, and distilled water; and an organic acid (such as citric acid or ascorbic acid) and distilled water. By combining organic acid, sodium phosphate dibasic, and sodium hydroxide, ointments of various pH levels are produced that activate the pH-sensitive tattoo inks individually or together. A further embodiment of the activating agent is a solution of Beckman Hydroxide Buffer at pH 12.45 being delivered to the tattoo site via use of an individual pre-moistened towelette wipe, spray-on mist, or ointment.

Yet another embodiment of the neutralizing agent for the selectively alterable intermittent tattoo ink is a solution of an organic acid and distilled water with a pH of 1 being delivered to the tattoo site by way of an individual pre-moistened wipe or towelette, spray-on mist, lotion or ointment.

Prepared pH ointment such as one consisting of an organic acid, sodium phosphate dibasic, and sodium hydroxide or Beckman Hydroxide Buffer at pH 12.45, can be applied topically to the skin over the tattoo to activate it. Pre-packed individual pre-moistened wipes or towelettes consisting of solutions of an organic acid, sodium phosphate dibasic and sodium hydroxide of various pH values or Beckman Hydroxide Buffer at pH 12.45, as well as a spray-on mist consisting of the same pH values can be applied to activate the tattoo ink. A prepared neutralizing ointment such as one consisting of an organic acid and water at pH 1 in a carrier base of Ultra-Myossage, or an organic acid and water delivered in a pre-moistened towelette or spray-on mist can be applied to deactivate the tattoo. Osmosis drives the H+ ions into the skin, activating the pH-sensitive pigments or, in the case with some of the pH-sensitive pigments listed above (or are listed in Table 1) which need a more basic environment to react, drive the H+ ions out of the skin, also activating the tattoo ink.

Referring to Table 1 there are listed various pH indicators, including descriptions of their color change, pH ranges and solubility/preparation. The preparation is not to be considered indicative of the actual ink formulation.

TABLE 1
Exemplary pH-sensitive tattoo ink compositions.

| Tattoo Ink | | Color | |
|---|---|---|---|
| Composition | pH Change | Change | Preparation |
| Quinaldine Red | 1.0-2.2 | Colorless | 1% in EtOH |
| | | to red | |
| 2,4-Dinitrophenol | 2.8-4.0 | Colorless | Saturated Water |
| | | to yellow | Solution |
| Ethyl Red | 4.0-5.8 | Colorless | 0.1 g in 50 ml |
| | | to red | MEOH + 50 ml |
| | | | water |
| p-Nitrophenol | 5.4-6.6 | Colorless | 0.1% in water |
| | | to yellow | |
| o-Cresolpthalein | 8.2-9.8 | Colorless | 0.04% in EtOH |
| | | to red | |
| Phenolphthalein | 8.2-10.0 | Colorless | 0.05 g in 50 ml |
| | | to pink | EtOH + 50 ml |
| | | | water |
| Ethyl-bis (2,4- | 8.4-9.6 | Colorless | Saturated solution |
| dimethylphenyl)acetate | | to blue | in 50% acetone |
| Thymolphthalein | 8.8-10.5 | Colorless | 0.04 g in 50 ml |
| | | to blue | EtOH + 50 ml |
| | | | water |
| 2,4,6-Trinitrotoluene | 11.5-13.0 | Colorless | 0.1-0.5% in EtOH |
| | | to orange | |
| 1,3,5-Trinitrobenzene | 12.0-14.0 | Colorless | 0.1-0.5% in EtOH |
| | | to orange | |

In the following exemplary formulae, percentages are by weight. Powdered pigments are pH active reagents selected from Table 1. An exemplary embodiment of the selectively alterable intermittent tattoo ink formulation is comprised of a mixture of glycerin, witch hazel, powered pigment and ethyl alcohol in the range from approximately 64% glycerin, 10% witch hazel, 26% powdered pigment, and no ethyl alcohol to: 52% glycerin, 23% witch hazel 17% powdered pigment, 8% ethyl alcohol.

The following formulations are alternative embodiments that are equally well suited and provide a suitable alternative for the commercial products Beckman's 12.45 pH buffer and Ultra-myosage lotion described herein.

An exemplary formulation of a corresponding acidic activator comprises approximately 90% distilled water and 10% citric acid by weight, which has a pH of approximately 1. This formulation is used in a spray-on mist and pre-moistened towelletes. An exemplary acidic lotion formation comprises approximately 48% glycerin, 34% citric acid, and 18% distilled water. This exemplary lotion formulation has a pH of approximately 3, as the glycerin acts as a buffer. Lower pH levels can be provided by using a formulation containing no or less glycerin.

An exemplary formulation of a corresponding alkaline activator is 89% distilled water, 9% sodium phosphate dibasic, and 2% sodium hydroxide, which has a pH of approximately 12.5. An exemplary alkaline lotion formulation comprises approximately 69% glycerin, 17% distilled water, and 14% sodium hydroxide, which has a pH of approximately 12.5.

The tattoo ink compositions that are either invisible (clear) or too light to clearly see while deactivated require that they be temporarily activated for the tattoo artist to use. Examples of such inks include those with pigments based on: Thymolphthalein, Phenolphthalein, o-Cresolphthalein, as well as other pigments. In these cases, the tattoo ink formulations are temporarily activated such as with the addition of approximately 0.03 grams of solution of 6% sodium hydroxide and 94% distilled water to 10 grams of ink. The solution temporarily raises the ink pH level to approximately 10. The temporarily activated ink is neutralized/deactivated by the body's natural buffering system and renders the tattoo invisible over time but is visible when the tattoo is being applied to the patient's skin.

In some tattoo ink formulations of the present disclosure, the acidic activator acts as a neutralizer for the inks activated on the alkaline side of the pH scale and vice a versa. Numerous pH activated powdered pigments can be made into selectively alterable intermittent tattoo inks using the above formulations and are activated by either the alkaline or acidic spray-on mist, pre-moistened towelletes, or lotions. In general the term lotion is used to describe a formulation which can be poured while an ointment does not pour. However, the configuration of a formulation as either a lotion or an ointment is primarily for user convenience.

Numerous modifications and alternative embodiments of the disclose tattoo ink compositions will be apparent to those skilled in the art in view of the foregoing description. For example, the pigments can be further reactive to temperature as well as other physiological changes and chemical concentrations in the skin. Furthermore, the activating formulations can be scented with specific scents, etc. Accordingly, this description is to be construed as illustrative only and is for the purpose of teaching those skilled in the art the best mode of carrying out the invention. Details of the structure may be varied substantially without departing from the spirit of the invention and the exclusive use of all modifications, which come within the scope of the appended claims, is reserved.

### UV Visible Tattoo Ink

The present disclosure generally provides tattoo ink compositions that are invisible to the naked eye until the tattoo is exposed to sunlight or another source of ultraviolet radiation, at which time the tattoo undergoes a photochemical change from clear or colorless to intensely colored, thereby producing a visible tattoo. Upon removal from exposure to UV, the tattoo again becomes invisible. By appropriate formulation, the tattoo can be made to return to clear and therefore become invisible almost immediately, or following a predetermined desired period of time.

The tattoo ink of the present disclosure is generally created by mixing a photochromic compound with a suitable carrier, such as the ink disclosed in U.S. Pat. No. 5,878,155, for example. The photochromic compound is selected from the spiropyrans and spiroxazine groups of the photochromic family of compounds, such as those disclosed in U.S. Pat. No. 5,581,090, for example. The spiropyrans and spiroxazine compounds normally appear colorless but undergo photochemical transformation and exhibit intense colors when exposed to UV radiation.

The spiropyrans or spiroxazine compounds may be added to the carrier in a proportion of about 0.1 to about 1.0 percent by weight. Higher concentrations yield more intense colors. Different color hues are obtained by mixing two or more spiropyrans or spiroxazine compounds in the carrier, as necessary or desired.

During application of the tattoo, it is necessary to expose the area being tattooed to UV radiation so that the design is visible to the tattooist. Upon exposure to UV radiation from a lamp or sunlight, for example, the tattoo becomes fully visible after only about five (5) seconds.

A commercially-available stabilizer may be added to the composition to reduce oxidation processes and thereby extend the useful life of the photochromic compound. Commercially-available stabilizers suitable for their performance under sunlight are Tinuvin 765 and Tinuvin 144 Hindred Amine Light Stabilizers, available from, e.g., the Additive Division of Ciba Geigy. The stabilizers may be added in a proportion of from about 2.5% up to about 3.0% by weight.

The photochromatic tattoo ink used with formulations of the invention can be applied using conventional tattooing methods and equipment, and when visible appears as a conventional tattoo. It can have the permanence, individuality, artistry and intricate detail of a conventional tattoo, but remains invisible until exposed to UV radiation. Thus, the person wearing the tattoo can dress in formal clothing or swim wear, etc., without displaying the tattoo, and when desired can activate the tattoo for display simply by exposing it to UV radiation. If desired, the tattoo can even be made to remain invisible when the person wearing the tattoo is exposed to sunlight or other source of UV radiation by covering the tattoo with an appropriate sun (UV) block.

### Producing Particles of the Formulation

Particles of the formulation can be produced in a number of different ways.

However, it is desirable that the particles be produced with certain specific characteristics. For example, it is desirable that the particles in a formulation all have the same size±20%, ±10%, ±5%, ±2%. It is also desirable that the process for producing the particles produce the particles without damaging the peptides of epitopes on the attenuated viruses in the particles. Such particles can be produced using a process referred to as “Flow Focusing” as disclosed within U.S. Pat. No. 6,116,516 issued Sep. 12, 2000 to Alfonso Ganan-Calvo, herein incorporated by reference.

Substantially any biocompatible polymer can be used in forming the particles. Those skilled in the art will appreciate that polymers can be selected from the group consisting of poly(lactic-co-glycolic acid) (PLGA), polycaprolactone, polyglycolide, polylactic acid, poly-3-hydroxybutyrate. Different polymer and different degree of polymerization and cross-linking can be used to obtain a desired release profile of the particles contents.

With respect to the peptide, the amino acid sequence in any given group of particles will be identical and will be chosen based on the particular pathogen of interest. Identical peptide sequences are produced and mixed with the desired polymer such as PLGA and the polymer with the peptides is extruded from a device (as disclosed within U.S. Pat. No. 6,116,516 which is incorporated herein by reference in its entirety) to produce particles.

In the present invention the epitope may consist only of an amino acid sequence, which could be produced from a virus. Viruses are grown either on primary cells such as chicken eggs (e.g., for influenza), or on continuous cell lines such as cultured human cells (e.g., for hepatitis A). Bacteria are grown in bioreactors (e.g., Haemophilus influenzae type b). Alternatively, a recombinant protein derived from the viruses or bacteria can be generated in yeast, bacteria, or cell cultures. After the antigen is generated, it is isolated from the cells used to generate it. A virus may need to be inactivated, possibly with no further purification required. Recombinant proteins need many operations involving ultrafiltration and column chromatography.

The vaccine may be formulated by adding adjuvant, stabilizers, and preservatives as needed to water, saline solution or other flowable liquid, all with or without ink which may be ink visible only under a black light. The adjuvant enhances the immune response of the antigen, stabilizers increase the storage life, and preservatives allow the use of multidose vials. Combination vaccines are harder to develop and produce, because of potential incompatibilities and interactions among the antigens and other ingredients involved.

Vaccine production techniques are evolving. Cultured mammalian cells are expected to become increasingly important, compared to conventional options such as chicken eggs, due to greater productivity and few problems with contamination. Recombination technology that produces genetically detoxified vaccine is expected to grow in popularity for the production of bacterial vaccines that use toxoids. Combination vaccines are expected to reduce the quantities of antigens they contain, and thereby decrease undesirable interactions, by using pathogen-associated molecular patterns. Groups of particles of the invention can be used to provide combination vaccines.

### Formulations

The compositions of the invention are especially useful for administration to an individual in need of immune stimulation (in the context of, for example, infectious disease, cancer, and allergy), and generally comprise a plurality of groups of microspheres of defined size comprising distinct antigen species as described herein, in a sufficient amount to modulate an immune response.

The compositions of the invention optionally comprise a pharmaceutically acceptable excipient, and may be in various formulations. As is well known in the art, a pharmaceutically acceptable excipient is a relatively inert substance that facilitates administration of a pharmacologically effective substance. For example, an excipient can provide form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery including nasal formulations are set forth in Remington's Pharmaceutical Sciences 19th Ed. Mack Publishing (1995).

Generally, these compositions are formulated for administration by inhalation or nasal application or by injection, e.g., intraperitoneally, intravenously, subcutaneously, intramuscularly, etc. Accordingly, these compositions are preferably combined with pharmaceutically acceptable vehicles such as saline, Ringer's solution, dextrose solution, and the like. The particular dosage regimen, i.e., dose, timing and repetition, will depend on the particular individual and that individual's medical history.

In some embodiments, more than one antigen(s) may be present in a composition. Compositions or formulations of the invention may be liquid dispersions where the carrier liquid is water and groups of particles provide the dispersion. A plurality of groups can provide at least two peptide species, at least 3, at least 4, at least 5, at least 10, at least 20, and usually not more than 50, not more than 40, not more than 30 peptide species. Such “cocktails”, as they are often denoted in the art, may be particularly useful in immunizing against pathogens present in different variants, e.g. HIV, rotavirus, influenza, etc. Alternatively, such formulations can provide information on developing a vaccine.

Generally, the efficacy of administering any of these compositions is adjusted by measuring any change in the immune response as described herein, or other clinical parameters.

In some embodiments, the invention provides compositions comprising polypeptides as described herein and an adjuvant whereby the polypeptide(s)/adjuvant are co-administered. The immunogenic composition may contain an amount of an adjuvant sufficient to potentiate the immune response to the immunogen. Adjuvants are known in the art and include, but are not limited to, oil-in-water emulsions, water-in oil emulsions, alum (aluminum salts), liposomes and microparticles including but not limited to, polystyrene, starch, polyphosphazene and polylactide/polyglycosides.

Other suitable adjuvants also include, but are not limited to, MF59, DETOX™ (Ribi), squalene mixtures (SAF-1), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875, as well as, lipid-based adjuvants and others described herein. For veterinary use and for production of antibodies in animals, mitogenic components of Freund's adjuvant (both complete and incomplete) can be used.

In some embodiments, the plurality of groups of microspheres or particles of defined size comprising distinct antigen species described herein can be administered in conjunction with one or more immunomodulatory facilitators. Thus, the invention provides compositions comprising plurality of microspheres of defined size comprising distinct antigen species and an immunomodulatory facilitator. As used herein, the term “immunomodulatory facilitator” refers to molecules which support and/or enhance immunomodulatory activity. Immunomodulatory facilitators include, but are not limited to, co-stimulatory molecules (such as cytokines, chemokines, targeting protein ligand, trans-activating factors, peptides, and peptides comprising a modified amino acid) and adjuvants (such as alum, lipid emulsions, and polylactide/polyglycolide microparticles).

The following excipients are commonly present in compositions to generate an immune response such as vaccine preparations. Aluminum salts or gels are added as adjuvants. Adjuvants are added to promote an earlier, more potent response, and more persistent immune response to the vaccine; they allow for a lower vaccine dosage. Antibiotics are added to some vaccines to prevent the growth of bacteria during production and storage of the vaccine. Egg protein is present in influenza and yellow fever vaccines as they are prepared using chicken eggs. Other proteins may be present. Formaldehyde is used to inactivate bacterial products for toxoid vaccines. Formaldehyde is also used to kill unwanted viruses and bacteria that might contaminate the vaccine during production. Monosodium glutamate (MSG) and 2-phenoxyethanol are used as stabilizers in a few vaccines to help the vaccine remain unchanged when the vaccine is exposed to heat, light, acidity, or humidity. Thimerosal is a mercury-containing preservative that is added to vials of vaccine that contain more than one dose to prevent contamination and growth of potentially harmful bacteria.

Many vaccines need preservatives to prevent serious adverse effects. Several preservatives are available, including thiomersal, phenoxyethanol, and formaldehyde. Thiomersal is more effective against bacteria, has better shelf life, and improves vaccine stability, potency, and safety, but in the U.S., the European Union, and a few other affluent countries, it is no longer used as a preservative in childhood vaccines, as a precautionary measure due to its mercury content. Although controversial claims have been made that thiomersal contributes to autism, no convincing scientific evidence supports these claims.

### Administration and Assessment of the Immune Response

The plurality of groups of microspheres or particles of the invention of defined size, where each group of particles defines a distinct antigen species composition can be administered in combination with other pharmaceutical and/or immunogenic and/or immunostimulatory agents and can be combined with a physiologically acceptable carrier thereof.

As with all immunogenic compositions, the immunologically effective amounts and method of administration of the particular formulation can vary based on the individual, what condition is to be treated and other factors evident to one skilled in the art. Factors to be considered include the antigenicity, route of administration and the number of immunizing doses to be administered. Such factors are known in the art and it is well within the skill of immunologists to make such determinations without undue experimentation. A suitable dosage range is one that provides the desired modulation of immune response to the antigen. Generally, a dosage range may be, for example, from about any of the following, referencing the amount of peptide in a dose exclusive of carrier: 0.01 to 100 μg, 0.01 to 50 μg, 0.01 to 25 μg, 0.01 to 10 μg, 1 to 500 μg, 100 to 400 μg, 200 to 300 μg, 1 to 100 μg, 100 to 200 μg, 300 to 400 μg, 400 to 500 μg. Alternatively, the doses can be about any of the following: 0.1 μg, 0.25 μg, 0.5 μg, 1.0 μg, 2.0 μg, 5.0 μg, 10 μg, 25 μg, 50 μg, 75 μg, 100 μg. Accordingly, dose ranges can be those with a lower limit about any of the following: 0.1 μg, 0.25 μg, 0.5 μg and 1.0 μg; and with an upper limit of about any of the following: 250 μg, 500 μg and 1000 μg. The absolute amount given to each patient depends on pharmacological properties such as bioavailability, clearance rate and route of administration.

The effective amount and method of administration of the particular formulation can vary based on the individual patient and other factors evident to one skilled in the art. Routes of administration include but are not limited to all types of injection including IV and IM, topical, dermal, transdermal, transmucosal, nasal, oral, epidermal, parenteral, gastrointestinal, and naso-pharyngeal and pulmonary, including transbronchial and transalveolar. The absolute amount given to each patient depends on pharmacological properties such as bioavailability, clearance rate and route of administration.

Parenteral routes of administration include but are not limited to electrical (iontophoresis) or direct injection such as direct injection into a central venous line, intravenous, intramuscular, intraperitoneal, intradermal, or subcutaneous injection. Compositions suitable for parenteral administration include, but are not limited, to pharmaceutically acceptable sterile isotonic solutions. Such solutions include, but are not limited to, saline and phosphate buffered saline for injection of the compositions.

Naso-pharyngeal and pulmonary routes of administration include, but are not limited to, inhalation, transbronchial and transalveolar routes. The invention includes compositions suitable for administration by inhalation including, but not limited to, various types of aerosols for inhalation, as well as powder forms for delivery systems. Devices suitable for administration by inhalation of include, but are not limited to, atomizers and vaporizers. Atomizers and vaporizers filled with the powders are among a variety of devices suitable for use in inhalation delivery of powders.

The methods of producing suitable devices for injection, topical application, atomizers and vaporizers are known in the art and will not be described in detail.

The above-mentioned compositions and methods of administration are meant to describe but not limit the methods of administering the compositions of the invention. The methods of producing the various compositions and devices are within the ability of one skilled in the art and are not described in detail here.

Analysis (both qualitative and quantitative) of the immune response to the subject compositions can be by any method known in the art, including, but not limited to, measuring antigen-specific antibody production (including measuring specific antibody subclasses), activation of specific populations of lymphocytes such as CD4+ T cells or NK cells, production of cytokines such as IFNγ, IL-2, IL-4, IL-5, IL-10 or IL-12 and/or release of histamine. Methods for measuring specific antibody responses include enzyme-linked immunosorbent assay (ELISA) and are well known in the art. Measurement of numbers of specific types of lymphocytes such as CD4+ T cells can be achieved, for example, with fluorescence-activated cell sorting (FACS). Serum concentrations of cytokines can be measured, for example, by ELISA. These and other assays to evaluate the immune response to an immunogen are well known in the art. See, for example, Selected Methods in Cellular Immunology (1980) Mishell and Shiigi, eds., W.H. Freeman and Co.

In some instances, a Th1 or Th2-type response is stimulated, i.e., elicited and/or enhanced. With reference to the invention, stimulating a Th1 or Th2-type immune response can be determined in vitro or ex vivo by measuring cytokine production from cells treated with a composition of the invention as compared to those treated with a conventional vaccine. Methods to determine the cytokine production of cells include those methods described herein and any known in the art. The type of cytokines produced in response to treatment indicates a Th1-type or a Th2-type biased immune response by the cells. As used herein, the term “Th1-type biased” cytokine production refers to the measurable increased production of cytokines associated with a Th1-type immune response in the presence of a stimulator as compared to production of such cytokines in the absence of stimulation. Examples of such Th1-type biased cytokines include, but are not limited to, IL-2, IL-12, and IFN-γ. In contrast, “Th2-type biased cytokines” refers to those associated with a Th2-type immune response, and include, but are not limited to, IL-4, IL-5, and IL-13. Cells useful for the determination of activity include cells of the immune system, primary cells isolated from a host and/or cell lines, usually APCs and lymphocytes.

### Delivery Systems

Tattoo systems can be used to deliver vaccine compositions to a patient. Tattoo systems are generally hand held devices that comprise one or more tattoo needles operably linked to a tattoo ink delivery system and one or more reservoirs of tattoo ink. Tattoo systems generally function by moving an actuator in a reciprocating motion that drives a tattoo needle into the skin of the patient, pushing the tattoo ink into the skin and depositing it there. Tattoo systems generally allow the user to control, e.g., the depth of penetration of the tattoo needle into the skin, the speed of the needle, and the force of application of the needle against the skin. Many tattoo systems are known in the art, and include, for example, tattoo systems used to create decorative tattoos on the skin of a subject, and medical tattoo systems, typically used in the field of radiation therapy to identify landmarks on a patient's skin so that subsequent doses of radiation treatment can be administered to the same location on a patient's body. Medical tattoo systems also include systems for drug delivery, e.g., for delivery of medicines to the skin of a patient. Exemplary tattoo systems include those disclosed in, e.g., U.S. Pat. Nos. 4,159,659, 6,626,927 and 6,685,719. Exemplary tattoo systems include rotary tattoo machines, coil tattoo machines, liner tattoo machines, shader tattoo machines, and color/saturation tattoo machines, any of which may be adapted for use with the compositions and methods of the present disclosure.

### Cosmetic Tattoo Systems

An exemplary tattoo system may comprise a frame assembly, an electromagnet assembly, an armature assembly, an interrupter switch assembly, a needle holder assembly, a needle guide or handpiece assembly, and a needle assembly, of which there may be several different types from which the operator selects one for performing particular tattooing functions. The needle assembly may comprise an elongated linear needle shaft and one or more small needles soldered to the end of the needle shaft opposite the end which is fitted in the needle holder block. Different tattooing needle assemblies may be used interchangeably, depending upon the nature of the work which is to be performed. Two commonly used tattooing needle assemblies are the outliner needle assembly and the shader needle assembly. In the outliner needle assembly, five needles are soldered together in parallel in a perfect circle. In the shader assembly, six needles are aligned side-by-side in line abreast and soldered together with adjacent needles contacting each other.

In use, an operator holds the tattooing device in one hand by a hand piece or handle. In some embodiments, the needles, or other selected needle group, are dipped in tattoo ink to prepare for application of the tattoo ink to the patient's skin. In some embodiments, tattoo ink is fed to the tattoo needle from a tattoo ink reservoir or cartridge in a continuous or semi-continuous manner. In some embodiments, the apparatus is energized by closing a foot switch that is provided in order to leave the operator's hands free for manipulation of the tattoo device. Once electric power is applied to the device, the electromagnets are energized and attract the armature bar. The attraction of the armature bar to the electromagnets separates the contacts of the interrupter switch, thereby de-energizing the electromagnets. The armature bar is then returned to the normal position by the armature support spring and the interrupter switch is again closed, restoring the continuity of the circuit to the electromagnets. The electromagnets thereby are alternately energized and de-energized repeatedly as long as the foot switch is closed. The armature bar oscillates and its oscillation causes the needle assembly to reciprocate in the needle guide assembly. The needle assembly, including the needle shaft and needles, is preferably made of stainless steel or other suitable stainless metal which may be sterilized.

### Surgical Tattoo Systems

In addition to the embodiments described above, the present disclosure generally provides surgical tattooing systems and methods that can be used to administer a vaccine composition to a patient. The following discussion includes a general description of the instrumentation utilized in applying a surgical tattoo followed by a description of a method or protocol utilizing the instrumentation.

A surgical tattoo ink dispensing instrument includes essentially three elements, namely, an ink cartridge, a leading cap, and a trailing cap. The ink cartridge defines an internal cavity that accommodates the sterile ink. The ink cartridge may have a generally frustoconical outer shape or configuration with an enlarged trailing end that gradually tapers to a smaller leading end. The leading end defines a narrowed distribution port that has an internal lumen communicating with the internal cavity of the ink cartridge.

The trailing end of the ink cartridge includes a circumferential collar to which a trailing cap is mounted. In one embodiment, the trailing cap includes a transverse outer wall which is joined to a circumferential collar of the ink cartridge by conventional means, including, for example adhesives, cements, and/or snap fit threaded means. In one embodiment, the trailing cap is joined to the ink cartridge through an ultrasonic welding technique. The trailing cap may define an internal circumferential stop that is received within the trailing end of the ink cartridge in sealing engagement therewith.

The trailing end cap defines an elongated length that facilitates functioning of the trailing end cap as a handle. In one embodiment, in use the operator holds the end cap in a manner similar to that of a writing implement, thus providing ease of control and a degree of familiarity to the operator.

The leading cap is placed on the ink cartridge prior to loading of the ink and preferably includes an internal stop that is received within the lumen of the port to fluidly seal the port at this end. The leading cap further defines an internal circumferential wall that is mounted about the port and preferably dimensioned so as to frictionally engage the port to thereby retain the end cap on the ink cartridge during assembly and shipping.

In some embodiments, the tattoo ink filled within the ink cartridge comprises a vaccine to be delivered to a patient. In some embodiments, the tattoo ink filled within the ink cartridge comprises a suitable tattoo ink, e.g., a selectively activatable tattoo ink, for use in tracking patients who have been vaccinated with a compound of the present disclosure. Any of a variety of suitable inks and/or vaccine compositions may be used in conjunction with the tattooing systems and methods discussed herein.

The components of the tattooing instrument may be fabricated, e.g., from a suitable polymeric material by known injection molding techniques. Ink cartridges may have some rigidity to minimize deformation thereof whereby the tattoo ink is gravity fed through a distribution port. Alternatively, an ink cartridge may be flexible to be squeezed in a manner to force the tattoo ink through the distribution port. In some embodiments, the trailing end cap is rigid.

Exemplary dimensions of the instrument may be as follows: length of trailing end cap: 2-3 inches, preferably about 2.5 inches; length of ink cartridge: 1.5-2.1 inches, preferably about 1.8 inches; diameter of trailing end cap: 0.250-0.450 inches, preferably about 0.350 inches. Other dimensions may be desirable based on the contemplated use of the device.

In one method of manufacture, the leading cap is mounted to the ink cartridge and the ink cartridge is filled with sterile tattoo ink. Thereafter, the trailing cap is then mounted to the ink cartridge and sealed through the ultrasonic weld joint. Thus the entire device is hermetically sealed, sterile and filled with the appropriate tattoo ink. This device can be prepackaged and shipped and delivered to the end user.

The ink dispensing instruments described above are intended for use in conjunction with a suitable tattoo needle, which may be secured to the device through any number of known conventional means. For example, the needle can be mounted about the distal port and connected therewith threaded means on the needle as internal threads that threadably engage corresponding threads of a cartridge, a bayonet coupling, a frictional engagement, etc. Alternatively, the needle is not required and the ink from the ink cartridge can be dispensed onto a needle point which is subsequently applied to the skin of the patient for tattooing.

In some embodiments, the trailing cap is eliminated. The ink cartridge may have an elongated portion or handle adjacent its trailing end and integrally attached thereto. The handle may include recessed grooves that accommodate the fingers of the user. The elongated portion of the handle enables the operator to hold the instrument in a manner similar to using a pencil or pen. This provides the user with a familiar feel that enhances control when applying the tattoo. In some embodiments, this instrument defines a length ranging from 4.00-4.50 inches, preferably about 4.25 inches.

In some embodiments, the device includes an ink cartridge defining a generally bulbous shape which is rounded at its proximal end. The distal end of the ink cartridge includes a distal port and has an end cap that is mounted to the distal port in the manner discussed above. The ink cartridge may comprise two components, namely, the ink base cartridge and a nozzle that defines the distal port and is ultrasonically welded to the base to connect the two components. The bulbous ink cartridge facilitates holding of the instrument between the index finger, middle finger, and the thumb, and provides a greater area to facilitate squeezing or deformation of the ink cartridge to expel the sterile tattoo ink.

### Surgical Tattooing Protocol

A surgical tattooing protocol for use with the instrumentation described above will now be discussed. First, a prepackaged ink dispensing instrument is provided and opened by tattooing personnel. A sterilized tattooing needle is mounted to the ink cartridge as discussed hereinabove. The needle is then applied to the skin of the patient. Next, the ink is dispensed or squeezed (or if gravity fed no squeezing is required) from the cartridge to deliver the ink through the distal port into the needle and then directed into the skin of the patient. After use, the needle and ink cartridge are discarded. In an alternate arrangement, a tattooing needle is not mounted to the ink cartridge, but, instead, is introduced to the skin. Thereafter, the tattoo ink is applied to the outer surface of the needle to travel along the needle and into the tissue.

It is contemplated that, in some embodiments, the dispensing instrument of the present disclosure will be individually wrapped in a sterile packet and boxed. The packets may be attached to each other, and, in such a case, an operator could pull off one packet at a time as needed. See, e.g., the dispensing system disclosed in U.S. Pat. Nos. 5,358,140 and 5,282,349, the contents of each being incorporated herein by reference. The instruments may be used for, e.g., safe and sterile application of tattoo ink to the skin of a patient for purposes of, e.g., administering a vaccine to a patient and/or marking a patient with an identifying marking. Other uses are contemplated as well.

In some embodiments, a tattoo system may be preloaded with permanent or semi-permanent tattoo ink. The preloaded instrument could be used to tattoo the skin of a patient and thereafter activated to deliver the tattoo ink to appropriately tattoo the patient for purposes of, e.g., tracking and/or identifying the patient following administration of a vaccine composition. After use, the instrument could be disposed of, although it is envisioned that in some embodiments the device could be sterilized for subsequent use(s).

The present disclosure thus contemplates the surgical tattooing methods and systems discussed above. The system assures that sterile tattoo ink is used for each individual patient.

There are several new delivery systems in development to make vaccine delivery more efficient. Methods include liposomes and ISCOM (immune stimulating complex). Other vaccine delivery technologies have resulted in oral vaccines. A polio vaccine was developed and tested by volunteer vaccinations with no formal training; the results were positive in that the ease of the vaccines increased dramatically. With an oral vaccine, there is no risk of blood contamination. Oral vaccines are likely to be solid which have proven to be more stable and less likely to freeze; this stability reduces the need for a “cold chain”: the resources required to keep vaccines within a restricted temperature range from the manufacturing stage to the point of administration, which, in turn, will decrease costs of vaccines.

A microneedle approach may be used, where the microneedle, which is “pointed projections fabricated into arrays that can create vaccine delivery pathways through the skin”. Microneedles (MN), as used herein, refers to an array comprising a plurality of micro-projections, generally ranging from about 25 to about 2000 μm in length, which are attached to a base support. An array may comprise 10², 10³, 10⁴, 10⁵ or more microneedles, and may range in area from about 0.1 cm² to about 100 cm². Application of MN arrays to biological membranes creates transport pathways of micron dimensions, which readily permit transport of macromolecules such as large polypeptides. The microneedle array may be formulated as a transdermal drug delivery patch. MN arrays can alternatively be integrated within an applicator device which, upon activation, can deliver the MN array into the skin surface, or the MN arrays can be applied to the skin and the device then activated to push the MN through the skin.

### Methods of the Invention

The invention also includes methods of modulating an immune response comprising administering an immunogenic formulation as described herein to an individual in an amount sufficient to modulate the immune response. Generally, the individual is in need of, or will be in need of, such modulation, due, for example, for a disease condition or being at risk of developing a disease condition. Examples of disease conditions include, but are not limited to, allergy, cancer, infectious diseases (such as viral or bacterial infection).

Methods of the invention include manufacturing particles of a biocompatible polymer having individual chemical species within the particle and then manufacturing groups of particles to create formulations and using those formulations to modulate an immune response within a subject, or, alternatively, using those formulations to study the immune system of a subject to obtain information in the development of a formulation to modulate an immune response in a subject.

A method of the invention includes administering a formulation of the invention to a patient such as a human patient and allowing the patient to generate an immune response to the formulation. The method may be carried out wherein a single formulation is administered to a patient wherein the single formulation is comprised of a plurality of groups of particles which are comprised of a biocompatible polymer and a chemical species such as an amino acid sequence which is an antigen specific to a pathogen. The particles within a given group may be identical and the particles within different groups are different with respect to the chemical species. The particles within all of the groups are within a size range in accordance with the invention such as a size range of 10 microns±20% to 25 microns±20%, or 13 microns to 17 microns in diameter±20%.

The methodology is intended to be carried out wherein the patient such as a human patient has not been previously exposed to any of the chemical species within the formulation administered to the patient. Thus, the method of the invention is generally not intended as a “booster” to supplement the immune response generated by a prior exposure of one or more of the antigens within the formulation. An aspect of the invention includes administering a formulation of the invention to a patient such as a human patient by a means such as injection, nasal spray or nasal drops and allowing the formulation to generate an immune response. The patient is generally a human patient believed to be at risk for an infection such as a viral or bacterial infection. Preferably, the patient has not been previously exposed artificially or naturally to pathogens (naturally) or antigens (artificially) which correspond to the antigens of the formulation of the invention. The reasoning as to why the method should be carried out as an initial and only exposure is that the concept of the invention relates at least in part to modulating or eliminating problems associated with immunodominance.

### System of Tattoo Marking

The present disclosure generally provides methods of vaccinating a subject and/or identifying the vaccinated subject through the use of one of more identifying markings that are applied to the subject's skin.

For example, in some embodiments a vaccine composition is formulated for delivery to a patient through the use of a tattoo system. First, the vaccine composition is administered to the patient by tattooing the patient with the vaccine composition, thereby administering the vaccine composition to the patient's skin. In some embodiments, the patient is subsequently tattooed using a tattoo ink that contains a visible or selectively activatable tattoo ink in order to create an identifying marking on the patient that can be used for identification purposes. For example, the patient may be tattooed with an identifying letter, number, symbol, or other device that identifies the patient based on the particular vaccine composition that was administered to the patient. Application of the identifying tattoo to the patient's skin allows clinical personnel to identify the particular vaccine composition that was administered to the patient. Following administration of the vaccine and application of the tattoo, the patient can be identified, and the patient's response to the vaccine can be tracked for clinical purposes.

### Administration of a Vaccine Composition and Application of an Identifying Tattoo at the Same Time

In some embodiments, a vaccine composition may be formulated to contain a tattoo ink that is visible or selectively activatable, and can thus be used to create an identifying marking on the patient at the same time that the vaccine composition is administered to the patient.

For example, in some embodiments, a vaccine composition is formulated to contain a visible or selectively activatable tattoo ink. The vaccine composition is loaded into a suitable tattoo device and the device is used to tattoo an identifying marking on the patient. Application of the tattoo ink administers the vaccine to the patient, while at the same time creating an identifying marking that can be used by clinical personnel to identify the particular vaccine composition that was administered to the patient. Following application of the tattoo, the patient can be identified, and the patient's response to the vaccine can be tracked for clinical purposes.

### Class I and Class II Epitopes

In one embodiment of the invention, only a single antigen species is present in a single particle or sphere. However, it is possible to include more antigen species in certain situations. As explained below different antigen species can be present in one particle when each species provides epitopes that are presented by different classes of MHC protein.

First it should be understood that an epitope, also known as antigenic determinant, is the part of an antigen that is recognized by an antigen receptor of the immune system, specifically by soluble antibodies, antibodies present on the surface of B cells, or T cells. Although epitopes are usually thought to be derived from non-self proteins, sequences derived from the host that can be recognized are also classified as epitopes, as well as non-proteinaceous antigens. The present invention is generally concerned with epitopes antigenic molecules from pathogens and more specifically pathogens such as bacteria and viruses that infect humans, and still more specifically those having an amino acid sequence limited to 8 to 12 amino acids

In humans the Major Histocompatibility Complex (MHC) is a set of molecules displayed on cell surfaces, which are integral to the antigen presentation machinery responsible for lymphocyte recognition and “antigen presentation”. The interaction between MHC molecules, antigen receptors and co-receptors controls mammalian immune responses, and provides a mechanism for distinguishing “self” and “non-self”. Both Class I and Class II MHC molecules belong to a group of molecules known as the Immunoglobulin Supergene Family, which includes immunoglobulin, T-cell receptors, CD4, CD8, and others.

Class I polypeptide molecules are composed of two polypeptide chains; one encoded by the MHC region and another (β2-microglobulin) that is encoded elsewhere. The MHC-encoded polypeptide is about 350 amino acids long and glycosylated, giving a total molecular weight of about 45 kDa. This polypeptide folds into three separate domains called alpha-1, alpha-2 and alpha-3. β2-microglobulin is a 12 kDa polypeptide that is non-covalently associated with the alpha-3 domain. Between the alpha-1 and alpha-2 domains lies a region bounded by a beta-pleated sheet on the bottom and two alpha helices on the sides. This region is capable of binding (via non-covalent interactions) a small peptide or epitope of about 8 to 12 amino acids. Included in the human class I proteins, also referred to as HLA, are HLA-A, HLA-B, HLA-C, and β₂-microglobulin.

Large protein antigens are cleaved within antigen presenting cells into small peptides, which may be from 8-12 amino acids in length. The small peptides that have an appropriate affinity for the MHC Class I protein (see, for example, Liao et al, (2011) PLoS One. 6(9):e25055, “Predicting peptide binding affinities to MHC molecules using a modified semi-empirical scoring function.”) and are complexed with the MHC protein, and are then transported to the cell surface, where they combine with protein components, i.e. coreceptors in the cellular membrane to form a complex that the T-cell recognizes. A CD8+ cytotoxic T-cell, for example, can be activated when the T cell antigen receptor on its surface binds to cognate epitopes complexed with class I mHC proteins on the surface of a cell, which can include without limitation antigen presenting cells.

In accordance with the invention, a single particle will generally only include one peptide or epitope that is presented by class I molecules present on the cell of interest. Such a peptide or epitope is referred to here as a class I peptide or a class I epitope.

Class II molecules are composed of two polypeptide chains. These polypeptides (alpha and beta) are about 230 and 240 amino acids long, respectively, and are glycosylated, giving molecular weights of about 33 kDa and 28 kDa. These polypeptides fold into two separate domains; alpha-1 and alpha-2 for the alpha polypeptide, and beta-1 and beta-2 for the beta polypeptide. Between the alpha-1 and beta-1 domains lies a region very similar to that seen on the class 1 molecule. This region, bounded by a beta-pleated sheet on the bottom and two alpha helices on the sides, is capable of binding (via non-covalent interactions) a small epitopes or peptide of about 8 to 12 amino acids, although a longer peptide can extend outside of the binding pocket (see, for example Fleckenstein et al. (1999) Semin Immunol. 11(6):405-16, “Quantitative analysis of peptide-MHC class II interaction”). These small peptides can be moved to the surface of the cell to form a complex recognizable by T-cells in a similar fashion to that described for Class 1 molecules as described above. Included in the human class II proteins are HLA-DPα, HLA-DPβ, HLA-DQα, HLA-DQβ, HLA-DRα and HLA-DRβ. These small peptides or epitopes are referred to here as class II peptides or class II epitopes.

While class I and class II molecules appear somewhat structurally similar and both present antigen to T-cells, their functions are distinct. Class I proteins are present on virtually all nucleated cells in the body, while class II protein expression is generally restricted to antigen presenting cells, e.g. dendritic cells, macrophages and B cells. Class I polypeptide molecules present antigen to cytotoxic T-cells (CTLs) in the context of CD8 co-receptor proteins, while class II polypeptide molecules present antigen to helper T-cells (TH-cells) in the context of CD4 co-receptor proteins.

Class I polypeptide molecules, which are present on the surface of virtually all nucleated mammalian cells, typically present “endogenous” antigens, which are fragments of proteins present within the cell. Examples include viral proteins or tumor proteins that are being actively expressed by the cell. Presentation of such antigens can indicate internal cellular alterations that if not contained could spread throughout the body. Hence, destruction of these cells by CTLs is advantageous to the body as a whole.

Class II polypeptide molecules present on APCs usually complex with “exogenous” antigens, which are not necessarily being expressed by the APC. Exogenous antigens are typically present in the extracellular space and are presented after being engulfed and processed by the APC. It may be noted that a viral protein could be endogenous to a cell infected by the virus, or exogenous to a cell that is exposed to a viral particle. The presentation of an antigen (class II peptide) by a class II polypeptide molecule usually stimulates T helper cells, which can, in turn, activate effector cells such as B cells (to produce antibodies), or CTLs.

For the case of the present invention, an immune response with memory is desired after a vaccination event, with or without the administration of a booster dose. Although the class-I epitopes administered in the micro particles provide sufficient information for activation of relevant T-cells to fight the infection of interest, an immune response from class-II epitopes may also be advantageous. In particular, a CD4 T-cell response to a “generic” epitope not specific for a CTL epitope may facilitate producing an anamnestic immune response to the CTL epitope. Formulations of the invention may include particles with multiple copies of one class I epitope and multiple copies of one class II epitope. In general, a single particle of the invention does not include multiple different class I epitopes.

An aspect of the invention is a particle comprised of an inert biocompatible polymer and an active ingredient which consists only of (a) multiple copies of a class I epitope and (b) multiple copies of a class II epitope. The particle has a size of about 12 to 18 microns or 15 microns±20%. The class I epitope and the class II epitope consists of 8 to 20 amino acids, or 8 to 12 amino acids or about 15 amino acids.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

In Example 1 10 micron diameter spherical particles 3, 3′, 3″, etc. comprised of poly(lactic-co-glycolic acid) (PLGA) are used. The particles had substantially the same diameter±10% or less. The particles were placed in a solution containing human dendrocytes 1 and 2. Photos were taken of the cells prior to (FIG. 1A), during (FIG. 1B) and (FIG. 1C) and after (FIG. 1D) the cells 1 and 2 consumed the particles. The particles 3, 3′, 3″, etc. were produced using a process as described within U.S. Pat. No. 6,116,516.

### Example 2

In Example 115 micron diameter spherical particles 3, 3′, 3″, etc. comprised of poly(lactic-co-glycolic acid) (PLGA) are used. The particles had substantially the same diameter±10% or less. The particles were placed in a solution containing human dendrocytes 1 and 2. Photos were taken of the cells prior to (FIG. 2A), during (FIG. 2B) and (FIG. 2C) and after (FIG. 2D) the cells 1 and 2 consumed the particles. The particles 3, 3′, 3″, etc. were produced using a process as described within U.S. Pat. No. 6,116,516.

Examples 1 and 2 show how groups of particles can be administered (placed in contact with dendrocytes) and used to determine the size of particles which the dendrocytes of the immune system readily consume. The results of Examples 1 and 2 indicate that for these dendrocytes, particles which are 10 microns in diameter are sufficiently small that multiple particles can be consumed by a single dendrocyte. The 15 micron particles of Example 2 indicate that, for these dendrocytes, only a single particle of 15 microns in diameter can be consumed. Thus, these two Examples indicate, for these dendrocytes, that the particles with a diameter of 15 microns can be used to present a single chemical species, and thereby reduce or eliminate immunodominance issues which might be created when multiple species are simultaneously presented by dendrocytes.

The formula for the volume of a sphere is (4/3)×π×(radius)³. The radius of the 10 micron sphere is 5 and 5³ is 125. The radius of the 15 microspheres is 7.5 and 7.5³ is 421.815. Thus, a relatively small increase in the diameter of a sphere makes a large increase in the volume of the sphere. The sphere volume is important in terms of the size of the particle that can undergo dendrocyte phagocytosis.

### Example 3

Synthesis of antigen containing microspheres. Microspheres of defined size, and containing a single peptide species were synthesized as described in Table 2 below.

TABLE 2

| Reagent Name | Supplier | Cat. No. | Purity |
|---|---|---|---|
| Resomer 502H | Boehringer Mannheim | 502H | 99% |
| D-(+)-Mannose | Sigma | M6020 | 98% |
| CMV pp65 peptide* | American Peptide | 305264 | 95% |
| Phosphate-buffered | Sigma | D8537 | 100% |
| saline (PBS) | | | |
| Acetone | Sigma | 270725 | ≧99.9% |

*Note:
Any peptide may be used in the synthesis.

CMV pp65 peptide was solubilized in PBS at 25 mg/ml (hereafter Reagent A; stored at 4 C). Mannose was solubilized in PBS at 200 mgs+400 uL PBS (hereafter Reagent B; stored at room temperature).

For 5 mls of formulation: a) Place 200 mgs of Resomer 502H in glass vial; b) Add 5.0 mL acetone and mix by rocking until Resomer is completely solubilized; c) Place vial in sonicator; d) During sonication, add 80 uL of Reagent A slowly, drop-wise; e) During sonication, add 20 uL of Reagent B slowly, drop-wise; f) Cap vial tightly and continue to sonicate.

The microspheres were formulated for use in cell cultures by suspension in appropriate culture medium. For use in pharmaceutical formulations the culture medium may be substituted with an appropriate excipient, e.g. normal saline, PBS, and the like.

In such formulations, 1 mg of the antigen containing microspheres is resuspended at a concentration (w/v) equal to 1 milligram/100 microliters in sterile tissue culture medium, and mixed well before use by flicking or sonication. The microspheres will settle within minutes so they must be re-mixed directly before using.

For phagocytosis experiments, 10 μl of resuspended microspheres were added to each well of as 24 well plate. In a 96 well plate, 2 μl t of resuspended microspheres are used.

### Example 4

### Assessment of Antigen Presentation

Preparation of Dendritic Cells from peripheral blood mononuclear cells (PBMC). Frozen PBMC cells were obtained from Cellular Technology Limited, from volunteer donors known to have been exposed to CMV, and to be HLA-A02. The volunteer's antigen presenting cells were known to be capable of presenting HCMV pp65 (NLVPMVATV) CMV epitope to T lymphocytes. Human PBMC (Peripheral Blood Mononuclear Cells) isolated from leukopacks and frozen in serum-free CTL-Cryo™ medium. These leukopacks were ethically collected from healthy donors tested for: HBsAg, HBcAb, HCV, HTLV I/II, and STS by serology; as well as HIV I, HCV, and WNV by NAT.

Cells were thawed and plated at a concentration of 1×10⁵ monocytes/well of a 24-well plate in medium supplemented with GM-CSF at a final concentration of 50 ng/ml and IL-4 at a final concentration of 100 ng/ml. Cells were maintained in culture at 37° C./5% CO₂ for 6 days; and used with 8 days after that point. Optical microscopy confirmed maturation of monocytes to dendrocytes capable of phagocytosis of 15 micron PLGA microspheres.

Presentation of antigen and stimulation of T cells was assessed by production of human γ-IFN, measured by ELISA. Mature dendrocytes were mixed with T-Lymphocytes and incubated for two hours with either free, soluble NLVPMVATV (at 10⁻⁹ M concentration) or 15 micron PLGA microspheres containing approximately 2% NLVPMVATV, prepared as described in Example 3. Production of γ-IFN was measured after 2 hours. The results are as shown in Table 3, demonstrating that 15 micron microspheres provide antigen that can be taken up by dendritic cells and presented to T cells.

TABLE 3

| Material Added To Cell Prep | ELISA Optical Density |
|---|---|
| Unstimulated Cells (no material added) | 0.055 |
| Incubated with free peptide | 0.612 |
| incubated with microspheres | 0.560 |

### Example 5

6-8 week old male C57B1/6 mice were subjected to intra-dermal (tail) injection with the H-2 Kb-restricted peptides SIINFEKL (OVA) and RGYVYQGL (VSV). Initial experiments demonstrated that free epitopes when injected with the adjuvants CpG and monophosphoryl lipid A (MPL) produced no measurable immune response when T-Cells harvested 14 days post inoculation were challenged with the same epitopes in-vitro with an Elispot assay evaluating an interferon gamma response.

### Example 6

The epitopes as described in Example 5 were encapsulated into PLGA microspheres as follows:

PLGA was prepared as a 4% (w/v) solution in acetone (typically, 200 mg of PLGA would be placed into an ultraclean borosilicate vial to which 5 mls of acetone would then be added). The vial was capped securely and placed on a rocker. The PLGA went completely into solution with gentle, constant agitation over 20 minutes. The peptide of choice was prepared as a 10 mg/ml solution in water. Mannose was prepared as a 100 mg/ml solution in water. The CpG ODN was prepared as a 10 mg/mL solution in water.

The solution of PLGA in acetone was next placed in a sonicator. Under constant sonication, the peptide was added. The amount of peptide varied, from 1 microliter (constituting one percent (1%) of maximal loading in the final microspheres by weight) to 100 microliters (constituting 100% of maximal loading). If necessary, an amount of water was then added to bring the total amount of aqueous solution added to 100 microliters (e.g., if 10 microliters of peptide was added, 90 microliters of water would then be added). While still under sonication, 50 microliters of mannose was added, followed by 50 microliters of the CpG ODN. This complete formulation was then sonicated for an addition five minutes.

Complete formulations were used in the Flow Focusing device as described in U.S. Pat. No. 6,116,516 (incorporated herein by reference) within one hour of their preparation. After passage through the Flow Focusing device, the resultant micro-particles collected were allowed to air dry overnight before harvesting and any further analysis and/or use in animal experiment procedures.

For the animal procedures, approximately 2.5 mgs of micro-particles were placed in a micro-centrifuge tube. On the day the animals were to be vaccinated, 112.5 microliters of water+0.1% Tween 20* was added to each tube, followed by the addition of 12.5 microliters of MPLA in DMSO. The micro-particles were briefly vortexed to re-suspend them in the liquid, and then 100 microliters of this suspension was injected into each animal (i.e., the resultant amount of micro-particles per animal was 2 mgs).

The Flow Focusing process was used to fabricate micro-spheres 13 microns in diameter so as to match the diameter of mouse antigen presenting cells as determined by light microscopy in preliminary experiments. This was done (a) to maximize the chance that only one sphere would be phagocytosed by any given antigen presenting cell and (b) to maximize the payload of epitope delivered to the antigen presenting cell per phaygocytosis event.

Liquid flow rate was set at 8.0 ml/hr. Gas pressure was 70 millibars. Needle ID was 0.006 inch, nozzle ID was 0.008 inch with 0.144 inch length, separation of needle to nozzle entrance was 0.006 inch. The needle is equivalent to the needle 60 of U.S. Pat. No. 6,116,516 and nozzle internal diameter is equivalent to the exit opening 68 of U.S. Pat. No. 6,116,516.

The peptide and other material are dissolved in acetone at a total concentration of about 5% w/v. The aerosol is generated at the top of an evaporation column where the wet droplet size is about 30 micrometers. After passing through an evaporation chamber (volume 2.7 liters, flow rate 12 l/min) most of the acetone is removed and the now dry particles pass through a Malvern Spraytech laser diffraction particle size analyzer, wherein they are measured.

After passing through the Spraytech, the particles were collected in an Anderson Cascade Impactor running at a flow rate of 15 l/min.

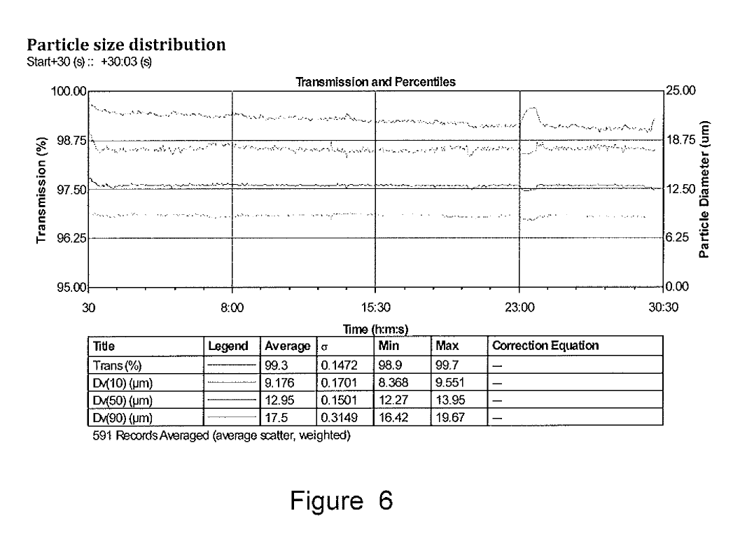

The plot shown in FIG. 6 is based on a 30 minute run shows a typical data set for this device. Particle size distribution is stable throughout the run with a mean size of 13 um. The distribution is further modified upon capture in the cascade impactor.

### Example 7

60 mice were intra-dermally injected with PLGA micro-spheres prepared as described in Example 6 containing escalating concentrations of OVA or VSV. The immune response was evaluated via anti-IFN-gamma Elispot assay 14 days after inoculation and the results as shown in Table 4 below:

TABLE 4

| | Sphere Loading As A | IF-Gamma response as |
|---|---|---|
| | Percent of Maximum | percent of positive |
| Epitope | Possible Loading | Elispot control |
| OVA (SIINFEKL) | 1% | 16 |
| VSV (RGYVYQGL) | 1% | 32 |

### Example 8

Because the 13 micron size of the fabricated micro-spheres was determined so as to ensure that any given antigen presenting cell was likely to only phagocytose and process epitopes from one sphere at a time, it was possible to design an experiment to see if a second epitope simultaneously loaded into a single microsphere in excess concentration could inhibit the presentation of the first loaded and also to examine if such an effect could be mitigated by only placing only one type of epitope in any given sphere. If two different sphere populations were given to the same mouse each loaded with a different epitope at a different concentration by weight, the large size of the spheres would make it unlikely that any single antigen presenting cell could phagocytose two spheres at once, making inhibition of one epitope by another in a single antigen presenting cell less likely.

This study was conducted to evaluate whether the addition of VSV in the same micro-particle with OVA will inhibit presentation of OVA peptide and thus reduce OVA-specific IFN-gamma and if placing OVA and VSV in separate micro particles would mitigate this inhibitory effect. 40 C57Bl/6 mice were inoculated with the micro-encapsulated epitopes as follows:

Three groups of 20 mice in each group were vaccinated with 2 mg epitope-loaded PLGA micro-particles by intra-dermal (tail) injection. 10 Mice in each group were inoculated with OVA and VSV epitopes in the same micro-sphere and 10 mice in each group were inoculated with OVA and VSV in separate spheres. An additional two mice in each 10 mouse test group served as controls receiving vehicle (PBS/0.1% Tween 20) only.

Responses to inoculation were determined by Elispot immune assay performed 14 days after initial injection evaluating harvested cells for IFN-gamma release on exposure to each of the two peptides injected and results are shown in Table 5 below:

TABLE 5

| OVA/VSV MICRO- | SAME SPHERE | OVA/VSV |
|---|---|---|
| SPHERE LOADED | OR DIFFERENT | IF-gamma |
| RATIO | SPHERES | RESPONSE RATIO |
| 1:1 | SAME SPHERES | 77 |
| 1:10 | SAME SPHERES | 49 |
| 1:100 | SAME SPHERES | 26 |
| 1:1 | DIFFERENT | 68 |
| | SPHERES | |
| 1:10 | DIFFERENT | 52 |
| | SPHERES | |
| 1:100 | DIFFERENT | 43 |
| | SPHERES | |

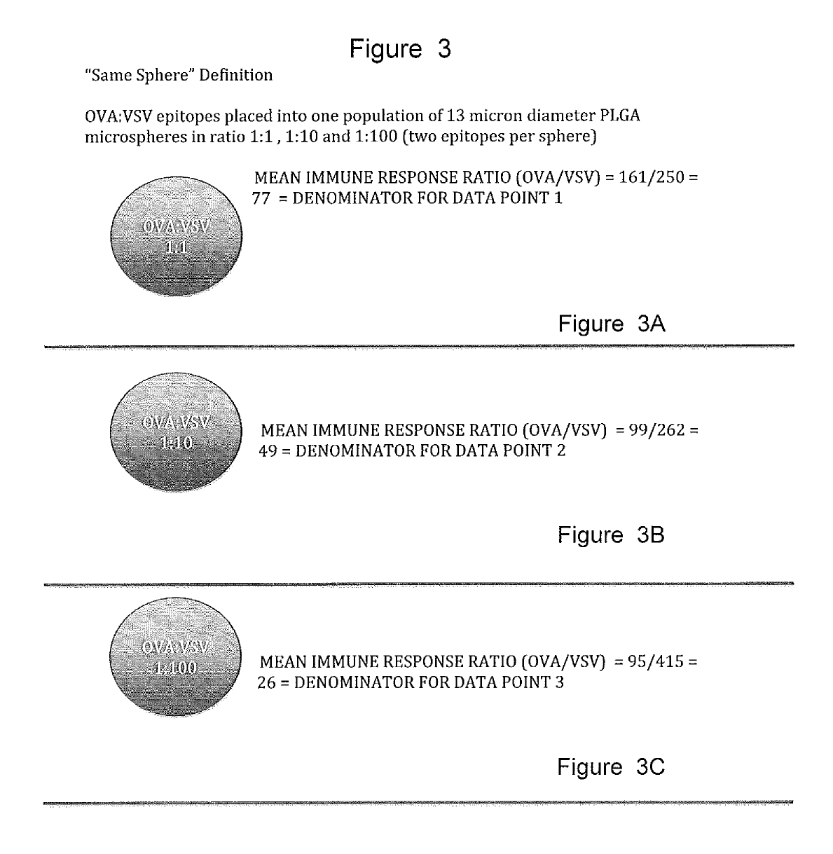

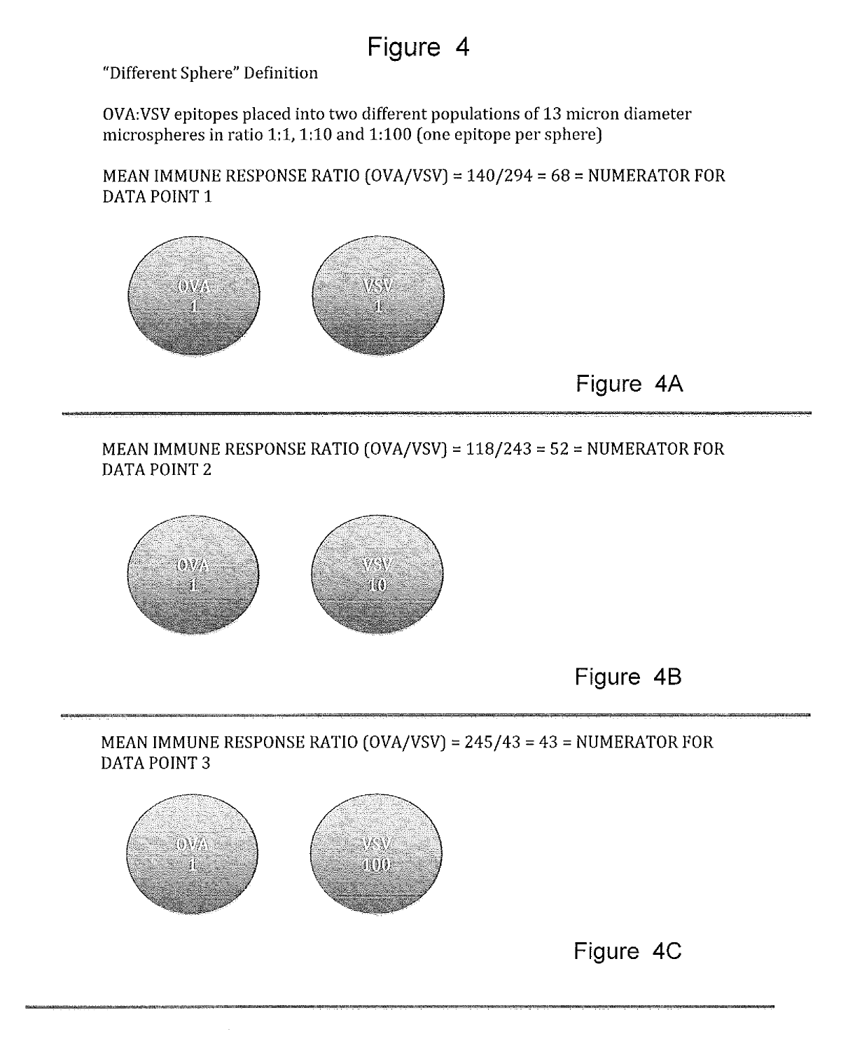

This data was used to calculate the relative improvement in immune response seen when the epitopes, at the same relative concentration ratios, are loaded in different spheres compared with the same spheres as shown in FIGS. 3 and 4 and summarized below in Table 6:

TABLE 6

| | OVA/VSV IMMUNE RESPONSE RATIO: |
|---|---|
| OVA/VSV RATIO | DIFFERENT SPHERES/SAME SPHERES |
| 1:1 | .88 |
| 1:10 | 1.06 |
| 1:100 | 1.65 |

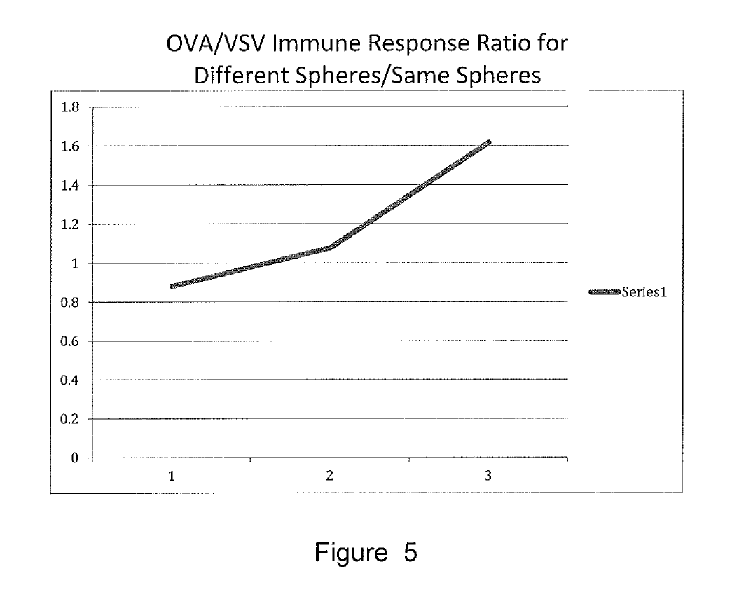

This data shows that when OVA and VSV epitopes at a ratio of 1:100 are placed in separate micro-spheres, a greater than 50% reduction in the inhibitory effect seen on OVA presentation by VSV can be seen when OVA and VSV at the same 1:100 relative amounts are each placed in different micro-spheres. The results of the groups of FIG. 3 are compared to the results of the groups of FIG. 4 were obtained and the ratios of these results are plotted in FIG. 5.

It is to be understood that this invention is not limited to the particular methodology, protocols, peptides, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A composition, comprising:
a first group of particles comprising a biocompatible polymer and a first active ingredient consisting only of a first peptide; and
a second group of particles comprising a biocompatible polymer and a second active ingredient consisting only of a second peptide different from the first peptide;
wherein each particle in the first group and the second group is substantially spherical, and has a diameter in a range of from 10 microns±20% to 25 microns±20%.

2. The composition of claim 2, wherein the second peptide has an amino acid sequence consisting of 8 to 12 amino acids.

3. The composition of claim 1, further comprising:
a third group of particles comprising a biocompatible polymer and a third active ingredient consisting only of a third peptide different from both the first and second peptides; and
wherein the biocompatible polymer is selected from the group consisting of poly(lactic-co-glycolic acid) (PLGA), polycaprolactone, polyglycolide, polylactic acid, poly-3-hydroxybutyrate.

4. The composition of claim 3, further comprising:
a plurality of additional groups of particles wherein additional peptides in each additional group are different from peptides in all other groups.

5. The composition of claim 4, further comprising:
a pharmaceutically acceptable carrier; and
an ink composition.

6. The composition of claim 5, further comprising:
an adjuvant and a pharmaceutically acceptable carrier;
wherein the ink is not visible to humans under normal ambient light.

7. The composition of claim 7, wherein ink fades over a period of twenty-four months or less, and all the in particles of the composition are 12 microns to 22 microns in diameter±20%.

8. A composition, comprising:
a first group of particles comprising an inert biocompatible polymer, and an active ingredient consisting only of (a) multiple copies of a first class I epitope and (b) multiple copies of a first class II epitope.

9. The composition of claim 8, further comprising:
a second group of particles comprising an inert a biocompatible polymer, and an active ingredient consisting only of multiple copies of a second class I epitope which is different from the first class I epitope or the first class II epitope.

10. The composition of claim 9, further comprising:
a third group of particles comprising an inert biocompatible polymer, and an active ingredient consisting only of multiple copies of a second class II epitope which is different from the first class I epitope or the first class II epitope.

11. The composition of claim 10, wherein each particle in the first, second and third group is substantially spherical, and has a diameter in a range of from 10 microns±20% to 25 microns±20%.

12. The composition of claim 10, wherein each class I epitope consist of 8 to 20 amino acids.

13. The composition of claim 12, wherein each class I epitope consists of 8 to 12 amino acids; and
wherein the biocompatible inert polymer is selected from the group consisting of poly(lactic-co-glycolic acid) (PLGA), polycaprolactone, polyglycolide, polylactic acid, poly-3-hydroxybutyrate.

14. The composition of claim 12, further comprising:
a plurality of additional groups of particles wherein additional peptides in each additional group are class I peptides and are different from peptides in all other groups; and
an adjuvant.

15. The composition of claim 14, wherein the adjuvant is selected from the group consisting of:
(a) spherical inert biocompatible particles; and
(b) spherical inert biocompatible particles and ink encapsulated in microparticles.

16. The composition of claim 15, further comprising:
a pharmaceutically acceptable carrier; and
wherein all the particles of the composition are 12 microns to 22 microns in diameter±20% and wherein the ink is only visible under a black light.

17. A method of treatment, comprising:
administering to a human patient a composition comprising (a) a first group of particles comprising a biocompatible polymer and a first chemical species, and (b) a second group of particles comprising a biocompatible polymer and a second chemical species, different from the first chemical species;
wherein each particle in the first group and the second group is substantially spherical, and has a diameter in a range of from 10 microns±20% to 25 microns±20%, and
further wherein the human patient has not been previously exposed to the first chemical species or the second chemical species.

18. The method of claim 17, further comprising:
administering to the patient an ink visible on skin of the patient.

19. The method of claim 18, wherein the ink is only visible under a black light.

20. The method of claim 19, wherein the ink is encapsulated in microparticles.
